(19) United States
(12) Patent Application Publication
(10) Pub. No.: US 2019/0326525 A1
(43) Pub. Date: Oct. 24, 2019

(54) ORGANIC ELECTROLUMINESCENT MATERIAL AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Bitnari KIM, Gyeonggi-do (KR); Dong-Hyung LEE, Gyeonggi-do (KR); Hyun KIM, Gyeonggi-do (KR)

(21) Appl. No.: 16/349,730

(22) PCT Filed: Dec. 5, 2017
(86) PCT No.: PCT/KR2017/014132

§ 371 Date: May 14, 2019

(30) Foreign Application Priority Data
Dec. 7, 2016 (KR) 10-2016-0166026

(51) Int. Cl. H01L 51/00; C09K 11/06; C09K 11/02; C07F 15/00; C07D 487/04

(52) U.S. Cl. CPC H01L 51/0085; C09K 11/06; C09K 11/02; C07F 15/0033; C07D 487/04; H01L 51/5012; C09K 2211/185; C09K 2211/1029; C09K 2211/1088; C09K 2211/1007; H01L 51/0072

(57) ABSTRACT

The present disclosure relates to an organic electroluminescent material comprising at least two types of compounds and an organic electroluminescent device comprising the same. The organic electroluminescent device having color purity superior to that of a conventional organic electroluminescent device can be provided by comprising the specific combination of the compounds of the present disclosure.

### TECHNICAL FIELD

The present disclosure relates to an organic electroluminescent material comprising at least two types of compounds and an organic electroluminescent device comprising the same.

### BACKGROUND ART

An electroluminescent device (EL device) is a self-light-emitting display device which has advantages in that it provides a wider viewing angle, a greater contrast ratio, and a faster response time. The first organic EL device was developed by Eastman Kodak in 1987, by using small aromatic diamine molecules and aluminum complexes as materials for forming a light-emitting layer (see Appl. Phys. Lett. 51, 913, 1987).

An organic electroluminescent device (OLED) changes electric energy into light by applying electricity to an organic electroluminescent material, and commonly comprises an anode, a cathode, and an organic layer formed between the two electrodes. The organic layer of the OLED may comprise a hole injection layer, a hole transport layer, a hole auxiliary layer, a light-emitting auxiliary layer, an electron blocking layer, a light-emitting layer (containing host and dopant materials), an electron buffer layer, a hole blocking layer, an electron transport layer, an electron injection layer, etc., if necessary. The materials used in the organic layer can be classified into a hole injection material, a hole transport material, a hole auxiliary material, a light-emitting auxiliary material, an electron blocking material, a light-emitting material, an electron buffer material, a hole blocking material, an electron transport material, an electron injection material, etc., depending on their functions. In the OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from an energy when the organic light-emitting compound returns to the ground state from the excited state.

The most important factor determining luminous efficiency in an OLED is light-emitting materials. The light-emitting materials are required to have the following features: high quantum efficiency, high mobility of an electron and a hole, and uniformity and stability of the formed light-emitting material layer. The light-emitting material is classified into blue, green, and red light-emitting materials according to the light-emitting color, and further includes yellow or orange light-emitting materials. Furthermore, the light-emitting material is classified into a host material and a dopant material in a functional aspect.

Generally, a device having excellent EL characteristics has a structure comprising a light-emitting layer made by doping a dopant to a host. When only one material is used as a light-emitting material, a problem arises in that the maximum emission wavelength moves toward a long wavelength and the color purity deteriorates due to intermolecular forces.

Iridium(III) complexes have been widely known as phosphorescent light-emitting materials, including bis(2-(2′-benzothienyl)-pyridinato-N,C-3′)iridium(acetylacetonate) [(acac)Ir(btp)₂], tris(2-phenylpyridine)iridium [Ir(ppy)₃] and bis(4,6-difluorophenylpyridinato-N,C2)picolinato iridium (Firpic) as red, green, and blue light-emitting materials, respectively.

Also, 4,4′-N,N′-dicarbazol-biphenyl (CBP) has been the most widely known phosphorescent host material. Recently, Pioneer (Japan) et al., developed a high performance organic electroluminescent device using bathocuproine (BCP) and aluminum(III) bis(2-methyl-8-quinolinate)(4-phenylphenolate) (BAlq), etc., which were known as hole blocking materials, as host materials.

However, although these conventional phosphorescent host materials provide good luminous characteristics, they have disadvantages in that the materials change when a high temperature deposition process is performed under vacuum due to low glass transition temperature and low thermal stability. Thus, the color purity of the device was still unsatisfactory.

Meanwhile, in order to implement a more vibrant display in the market of the current FHD (Full-HD), advanced UHD (Ultra-HD) and more advanced display, it should be possible to express various colors. There are a number of criteria that can be defined, of which color space is representative. The color space refers to an area of a color that can be combined with each vertex of red, green, and blue, which are the three primary colors of light. Recently, many electronic panel companies are making organic electroluminescent devices by using three colors of blue, green, and red, and these three colors are combined to realize various colors of displays that are currently in use. The display can implement only the colors that can be combined with one another at the three vertexes of blue, green, and red. Thus, for a more colorful implementation, these three color vertexes must reach their respective color wavelengths as much as possible.

Korean Patent Application Laid-Open Nos. 2010-0014633, 2011-0074538 and 2015-0003670 disclose an organic electroluminescent device comprising an iridium complex having a ligand of a phenylquinoline as a dopant. Also, Korean Patent No. 1082144 and Korean Patent Application Laid-Open No. 2016-0039561 disclose an organic electroluminescent device comprising an indolocarbazole derivative as a host. However, the aforementioned documents do not specifically disclose an organic electroluminescent device comprising an iridium complex having a ligand of a phenylquinoline as a dopant, and a benzoindolocarbazole derivative as a host.

### DISCLOSURE OF THE INVENTION

### Problems to be Solved

The objective of the present disclosure is to provide an organic electroluminescent material capable of producing an organic electroluminescent device having color purity superior to that of a conventional organic electroluminescent device.

### Solution to Problems

As a result of intensive studies to solve the technical problems, the present inventors have focused on a dark red light-emitting device having a longer wavelength. Thereafter, the present invention was completed in such a direction that the color reproduction range may be increased by drawing the wavelength band in the red light-emission to longer wavelength as much as possible. Although the characteristics of the dopant material of host and dopant materials highly influence the wavelength band, it is possible to further optimize the luminous characteristics by using a host material suitable for the dopant material. In order to represent the longer wavelength, the dopant emitting the specific light has to be smaller in the HOMO-LUMO bandgap. In the present disclosure, a host having a small energy bandgap that allows energy transfer with the specific dopant is searched to provide better color space characteristics. Specifically, a combination of a host of a benzoindolocarbazole and a dopant comprising an isoquinoline structure can achieve the above objective. More specifically, the above objective can be achieved by an organic electroluminescent material comprising a compound represented by the following formula 1 and a compound represented by the following formula 2. The compound represented by formula 1 may be comprised as a dopant, and the compound represented by formula 2 may be comprised as a host.

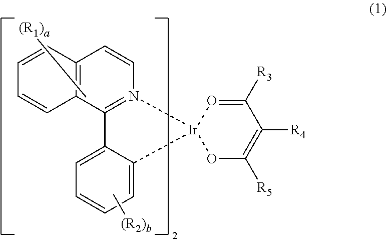

Wherein

R₁ and R₂, each independently, represent hydrogen, deuterium, a substituted or unsubstituted (C1-C10)alkyl, a substituted or unsubstituted (C2-C10)alkenyl, or a substituted or unsubstituted (C6-C30)aryl, with a proviso that both R₁ and R₂ are not hydrogen,

R₃ to R₅, each independently, represent hydrogen, deuterium, a substituted or unsubstituted (C1-C10)alkyl, or a substituted or unsubstituted (C6-C30)aryl, and

a represents an integer of 1 to 6, and b represents an integer of 1 to 4, where if a and b, each independently, are an integer of 2 or more, each of R₁ and R₂ may be the same or different.

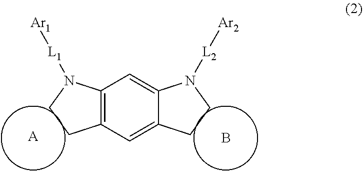

Wherein

A ring and B ring, each independently, represent a substituted or unsubstituted benzene ring, or a substituted or unsubstituted naphthalene ring, with a proviso that at least one of A ring and B ring is a substituted or unsubstituted naphthalene ring,

Ar₁ and Ar₂, each independently, represent a substituted or unsubstituted (C6-C30)aryl, or a substituted or unsubstituted nitrogen-containing (8- to 30-membered)heteroaryl,

L₁ and L₂, each independently, represent a single bond, a substituted or unsubstituted (C6-C30)arylene, or a substituted or unsubstituted (3- to 30-membered)heteroarylene, and

the heteroaryl(ene) contains at least one heteroatom selected from B, N, O, S, Si, and P.

### Effects of the Invention

According to the present disclosure, an organic electroluminescent device having excellent color purity can be provided.

### BRIEF DESCRIPTION OF THE FIGURES

FIG. 1 illustrates an NTSC Color Space of Table 1 of the present disclosure as a CIE 1931 Chromaticity Diagram.

### EMBODIMENTS OF THE INVENTION

Hereinafter, the present disclosure will be described in detail. However, the following description is intended to explain the disclosure, and is not meant in any way to restrict the scope of the disclosure.

The term “an organic electroluminescent material” in the present disclosure means a material that may be used in an organic electroluminescent device, and may comprise at least one compound. If necessary, the organic electroluminescent material may be comprised in any layers constituting an organic electroluminescent device. For example, the organic electroluminescent material may be a hole injection material, a hole transport material, a hole auxiliary material, a light-emitting auxiliary material, an electron blocking material, a light-emitting material, an electron buffer material, a hole blocking material, an electron transport material, an electron injection material, etc.

The organic electroluminescent material of the present disclosure may comprise at least one compound represented by formula 1, and at least one compound represented by formula 2. The compound of formula 1 and the compound of formula 2 may be included in the light-emitting layer, but are not limited thereto. In this case, the compound of formula 1 may be included as a dopant, and the compound of formula 2 may be included as a host.

In formula 1, R₁ and R₂, each independently, represent hydrogen, deuterium, a substituted or unsubstituted (C1-C10)alkyl, a substituted or unsubstituted (C2-C10)alkenyl, or a substituted or unsubstituted (C6-C30)aryl; and in one embodiment of the present disclosure, hydrogen, or a substituted or unsubstituted (C1-C6)alkyl; with a proviso that both R₁ and R₂ are not hydrogen. In another embodiment of the present disclosure, R₁ represents hydrogen, or a substituted or unsubstituted (C1-C6)alkyl, and R₂ represents a substituted or unsubstituted (C1-C6)alkyl. For example, R₁ may represent hydrogen, a methyl, an iso-propyl, an n-butyl, an iso-butyl, or a sec-butyl, and R₂ may represent a methyl, an iso-butyl, or a tert-butyl.

In formula 1, R₃ to R₅, each independently, represent hydrogen, deuterium, a substituted or unsubstituted (C1-C10)alkyl, or a substituted or unsubstituted (C6-C30)aryl; and in one embodiment of the present disclosure, hydrogen, a substituted or unsubstituted (C1-C6)alkyl, or a substituted or unsubstituted (C6-C25)aryl. In another embodiment of the present disclosure, R₃ and R₅, each independently, represent a substituted or unsubstituted (C1-C6)alkyl, or a substituted or unsubstituted (C6-C18)aryl; and R₄ represents hydrogen, a substituted or unsubstituted (C1-C6)alkyl, or a substituted or unsubstituted (C6-C18)aryl. In addition, R₃ and R₅ may be the same as each other. For example, R₃ and R₅, each independently, may represent a methyl, an iso-propyl, an iso-butyl, a sec-butyl, a 3-pentyl or a phenyl, and R₄ may represent hydrogen, a methyl or a phenyl.

In formula 1, a represents an integer of 1 to 6, and b represents an integer of 1 to 4, where if a and b, each independently, are an integer of 2 or more, each of R₁ and R₂ may be the same or different. In one embodiment of the present disclosure, a and b, each independently, represent 1 or 2, where if a and b, each independently, are 2, each of R₁ and R₂ may be the same or different.

In formula 2, A ring and B ring, each independently, represent a substituted or unsubstituted benzene ring, or a substituted or unsubstituted naphthalene ring, with a proviso that at least one of A ring and B ring is a substituted or unsubstituted naphthalene ring. In one embodiment of the present disclosure, any one of A ring and B ring may represent a substituted or unsubstituted benzene ring, and the other may represent a substituted or unsubstituted naphthalene ring. A ring and B ring, each independently, may represent an unsubstituted benzene ring, or a naphthalene ring unsubstituted or substituted with a phenyl(s).

In formula 2, Ar₁ and Ar₂, each independently, represent a substituted or unsubstituted (C6-C30)aryl, or a substituted or unsubstituted nitrogen-containing (8- to 30-membered)heteroaryl. Any one of Ar₁ and Ar₂ may represent a substituted or unsubstituted (C6-C30)aryl, and the other may represent a substituted or unsubstituted nitrogen-containing (8- to 30-membered)heteroaryl. In one embodiment of the present disclosure, Ar₁ and Ar₂, each independently, represent a substituted or unsubstituted (C6-C25)aryl, or a substituted or unsubstituted nitrogen-containing (8- to 25-membered)heteroaryl; and in another embodiment of the present disclosure, represent a substituted or unsubstituted (C6-C18)aryl, or a substituted or unsubstituted nitrogen-containing (8- to 18-membered)heteroaryl. For example, Ar₁ and Ar₂, each independently, represent an unsubstituted phenyl; an unsubstituted naphthyl; a quinazolinyl substituted with a phenyl(s); or a quinoxalinyl unsubstituted or substituted with a phenyl(s), a biphenyl(s), a naphthyl(s), a naphthylphenyl(s), and/or a dimethylfluorenyl(s).

In formula 2, L₁ and L₂, each independently, represent a single bond, a substituted or unsubstituted (C6-C30)arylene, or a substituted or unsubstituted (3- to 30-membered)heteroarylene; in one embodiment of the present disclosure, a single bond, a substituted or unsubstituted (C6-C25)arylene, or a substituted or unsubstituted (5- to 25-membered)heteroarylene; in another embodiment of the present disclosure, a single bond, a substituted or unsubstituted (C6-C18)arylene, or a substituted or unsubstituted (5- to 18-membered)heteroarylene; and, for example, a single bond, a phenylene, a naphthylene, or a pyridinylene.

In formulas 1 and 2, the heteroaryl(ene) contains at least one heteroatom selected from B, N, O, S, Si, and P; in one embodiment of the present disclosure, may contain at least one heteroatom selected from N, O, and S; and in another embodiment of the present disclosure, may contain a nitrogen atom(s).

In one embodiment of the present disclosure, formula 2 may be represented by the following formula 3.

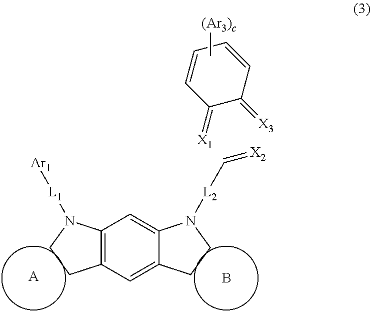

In formula 3, any one of A ring and B ring represents a substituted or unsubstituted naphthalene ring, and the other represents a substituted or unsubstituted benzene ring. In one embodiment of the present disclosure, any one of A ring and B ring represents a naphthalene ring unsubstituted or substituted with a phenyl(s), and the other represents an unsubstituted benzene ring.

In formula 3, X₁ to X₃, each independently, represent CR₆ or N, with a proviso that at least one of X₁ to X₃ represents N; in one embodiment of the present disclosure, at least two of X₁ to X₃ may represent N; and in another embodiment of the present disclosure, two of X₁ to X₃ may represent N. For example, X₁ may represent N; any one of X₂ and X₃ may represent N; and the other one of X₂ and X₃ may represent CR₆.

Herein, R₆ represents hydrogen, or a substituted or unsubstituted (C6-C30)aryl; in one embodiment of the present disclosure, a substituted or unsubstituted (C6-C25)aryl; in another embodiment of the present disclosure, a substituted or unsubstituted (C6-C18)aryl; and, for example, a phenyl, a naphthyl, a biphenyl, or a naphthylphenyl.

In formula 3, Ar₃ represents hydrogen, a substituted or unsubstituted (C1-C30)alkyl, a substituted or unsubstituted (C6-C30)aryl, or a substituted or unsubstituted (3- to 30-membered)heteroaryl; in one embodiment of the present disclosure, hydrogen, a substituted or unsubstituted (C1-C20)alkyl, a substituted or unsubstituted (C6-C25)aryl, or a substituted or unsubstituted (3- to 25-membered)heteroaryl; in another embodiment of the present disclosure, hydrogen, or a substituted or unsubstituted (C6-C18)aryl; and, for example, hydrogen, a phenyl, a naphthyl, a biphenyl, a naphthylphenyl, or a dimethylfluorenyl.

In formula 3, c represents an integer of 1 to 4; in one embodiment of the present disclosure, may represent 1 or 2; and in another embodiment of the present disclosure, may represent 1. If c is an integer of 2 or more, each of Ar₃ may be the same or different.

In formula 3, Ar₁, L₁, and L₂ are as defined in formula 2.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, iso-propyl, n-butyl, iso-butyl, tert-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered) heterocycloalkyl” is meant to be a cycloalkyl having 3 to 7, preferably 5 to 7, ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, in which the number of the ring backbone carbon atoms is preferably 6 to 25, more preferably 6 to 18. The above aryl(ene) may be partially saturated, and may comprise a spiro structure. The above aryl may include phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. The term “(3- to 30-membered)heteroaryl(ene)” is an aryl having 3 to 30 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, quinoxalinyl, naphthyridinyl, carbazolyl, benzocarbazolyl, dibenzocarbazolyl, phenoxazinyl, phenothiazinyl, phenanthridinyl, benzodioxolyl, and dihydroacridinyl. Furthermore, “halogen” includes F, Cl, Br, and I.

Herein, “substituted” in the expression “substituted or unsubstituted” means that a hydrogen atom in a certain functional group is replaced with another atom or another functional group, i.e. a substituent. The substituents of the substituted alkyl, the substituted alkenyl, the substituted aryl(ene), the substituted heteroaryl(ene), the substituted benzene ring, and the substituted naphthalene ring in R₁ to R₆, Ar₁ to Ar₃, L₁, L₂, A ring, and B ring of formulas 1 to 3, each independently, may be at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (C6-C30)aryl, a (5- to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl; in one embodiment of the present disclosure, at least one (C6-C25)aryl; in another embodiment of the present disclosure, at least one (C6-C18)aryl; and, for example, at least one selected from the group consisting of phenyl, biphenyl, naphthyl, naphthylphenyl and dimethylfluorenyl.

The compound represented by formula 1 includes the following compounds, but is not limited thereto.

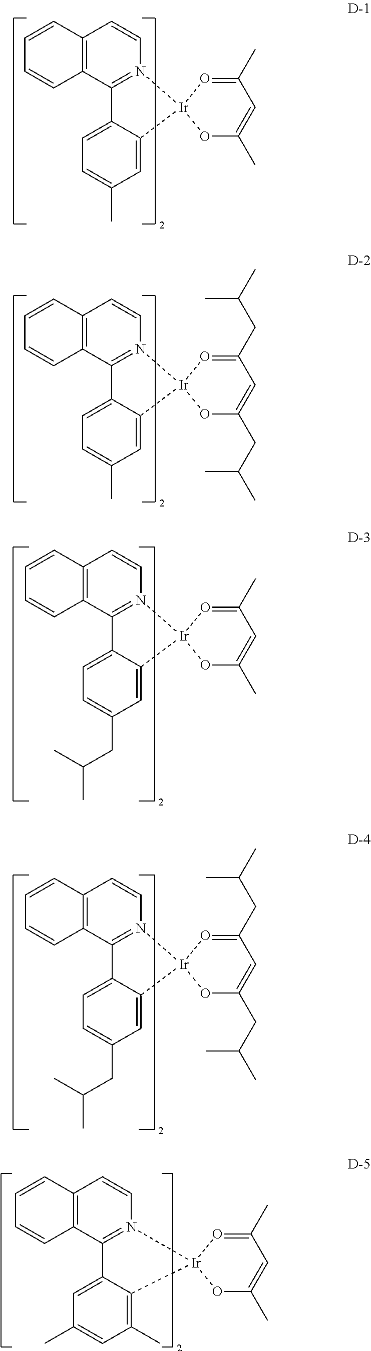

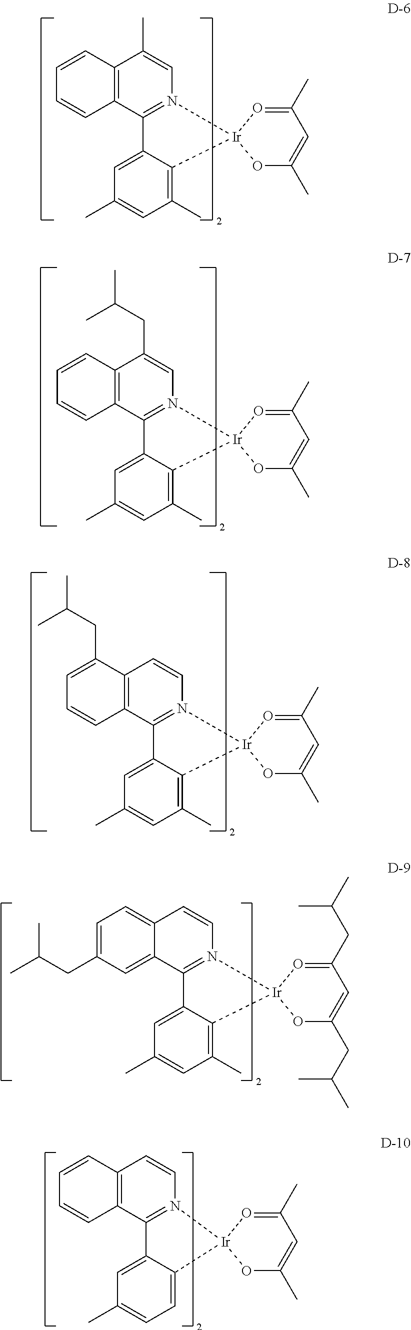

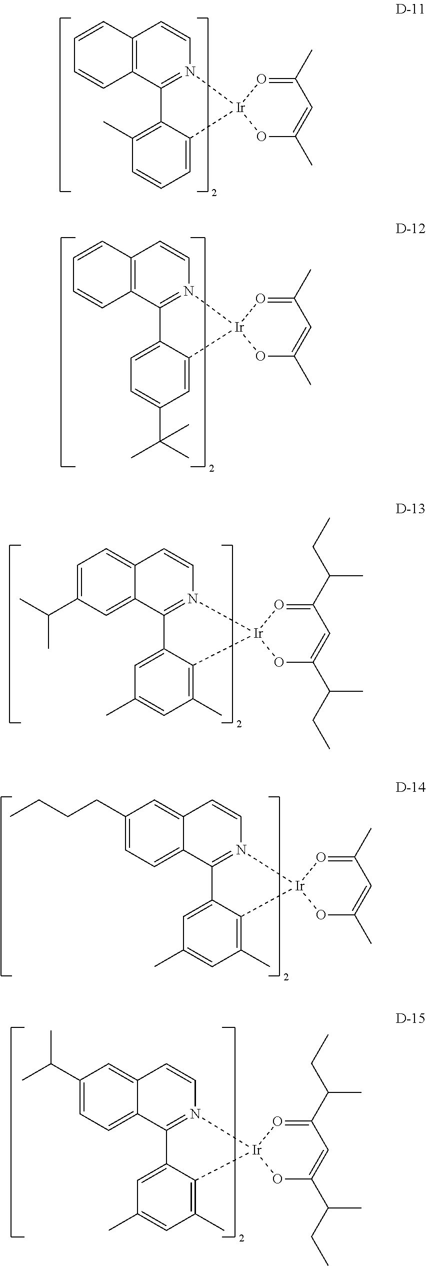

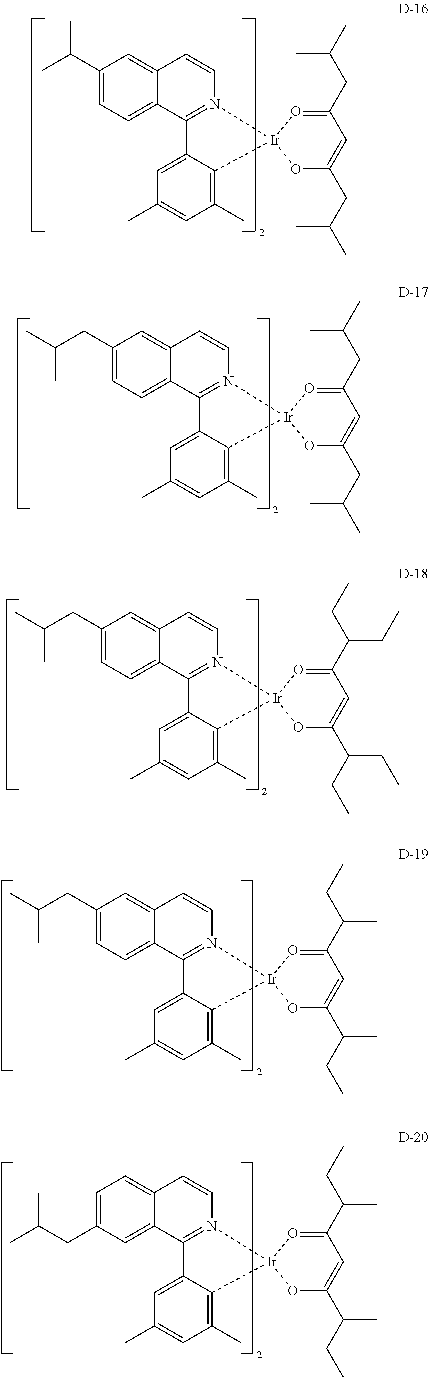

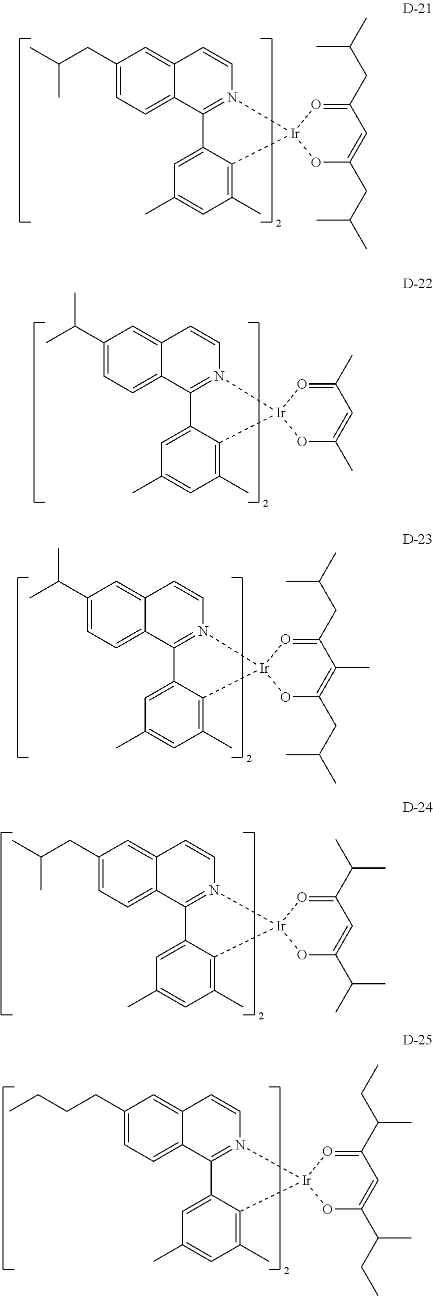

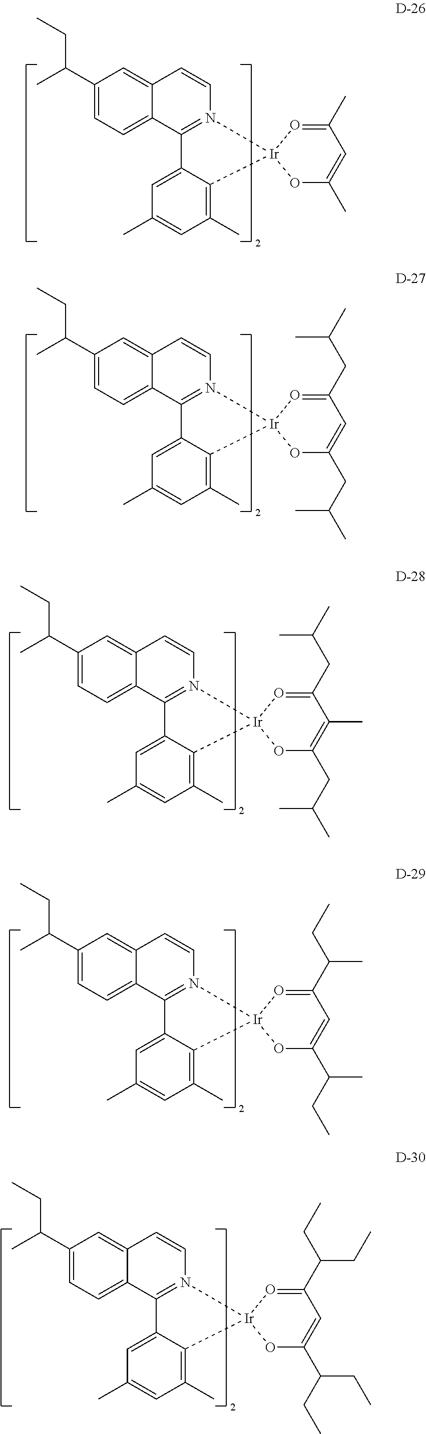

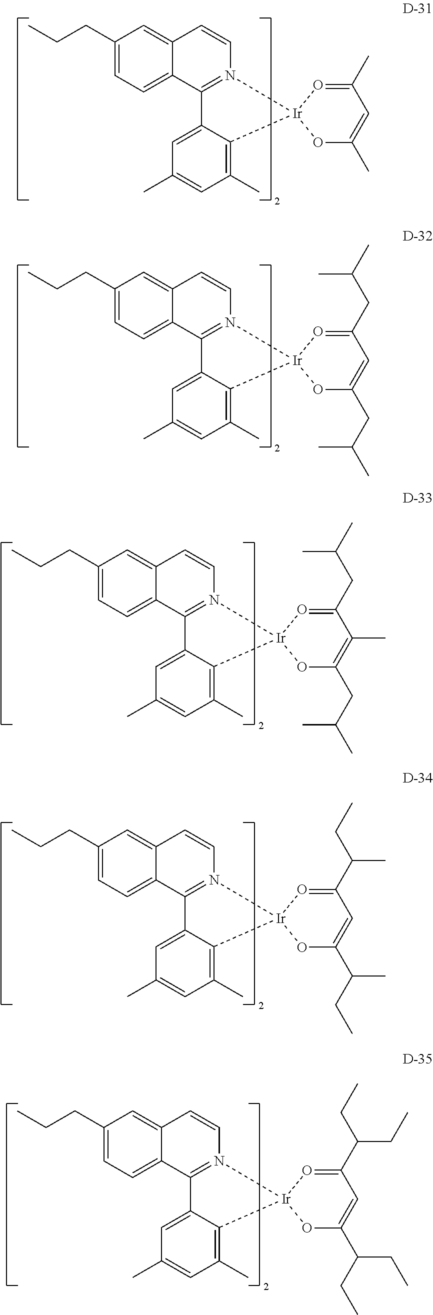

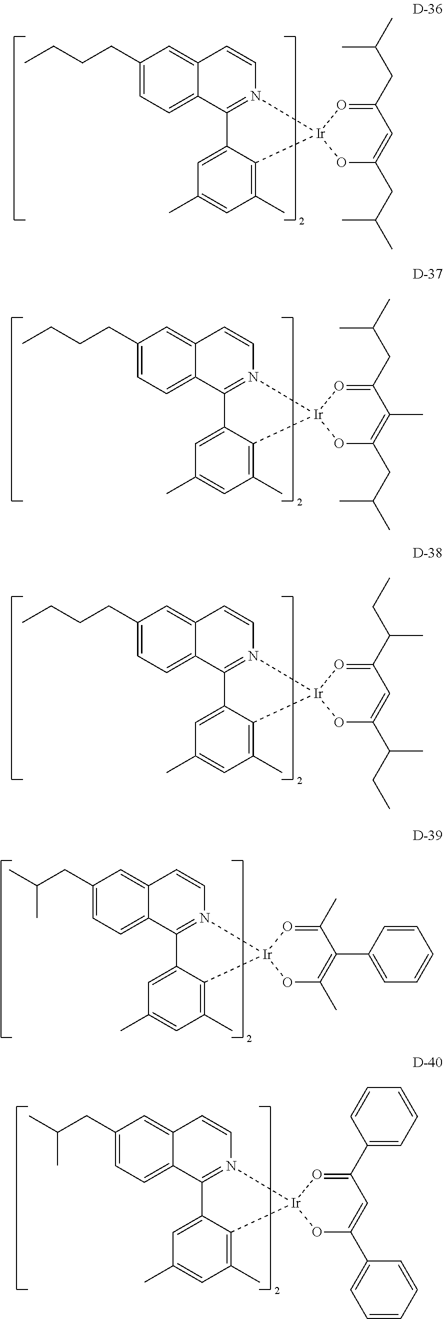

The compound represented by formula 2 includes the following compounds, but is not limited thereto.

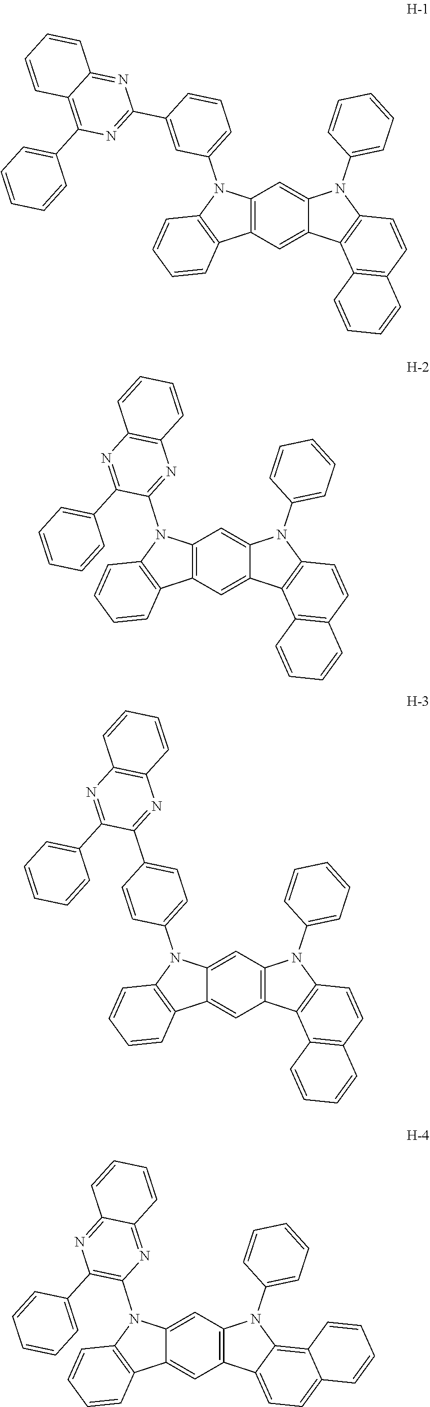 [SMILES: c1ccc(-c2nc(-c3cccc(-n4c5ccccc5c5cc6c7c8ccccc8ccc7n(-c7ccccc7)c6cc54)c3)nc3ccccc23)cc1 ; c1ccc(-c2nc3ccccc3nc2-n2c3ccccc3c3cc4c5c6ccccc6ccc5n(-c5ccccc5)c4cc32)cc1 ; c1ccc(-c2nc3ccccc3nc2-c2ccc(-n3c4ccccc4c4cc5c6c7ccccc7ccc6n(-c6ccccc6)c5cc43)cc2)cc1 ; c1ccc(-c2nc3ccccc3nc2-n2c3ccccc3c3cc4c5ccc6ccccc6c5n(-c5ccccc5)c4cc32)cc1]

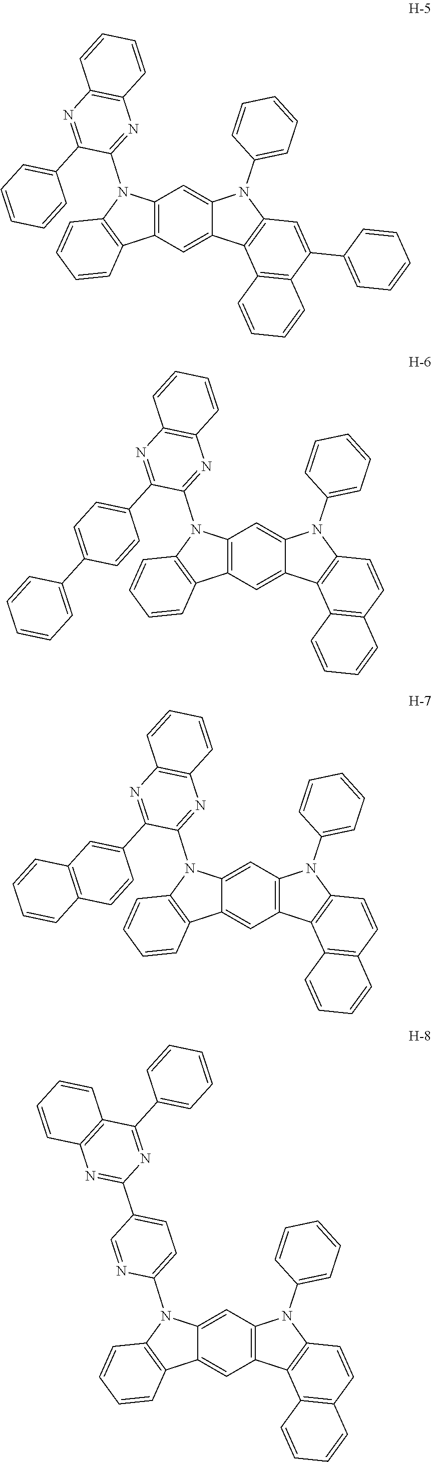 [SMILES: c1ccc(-c2nc3ccccc3nc2-n2c3ccccc3c3cc4c5c6ccccc6c(-c6ccccc6)cc5n(-c5ccccc5)c4cc32)cc1 ; c1ccc(-c2ccc(-c3nc4ccccc4nc3-n3c4ccccc4c4cc5c6c7ccccc7ccc6n(-c6ccccc6)c5cc43)cc2)cc1 ; c1ccc(-n2c3cc4c(cc3c3c5ccccc5ccc32)c2ccccc2n4-c2nc3ccccc3nc2-c2ccc3ccccc3c2)cc1 ; c1ccc(-c2nc(-c3ccc(-n4c5ccccc5c5cc6c7c8ccccc8ccc7n(-c7ccccc7)c6cc54)nc3)nc3ccccc23)cc1]

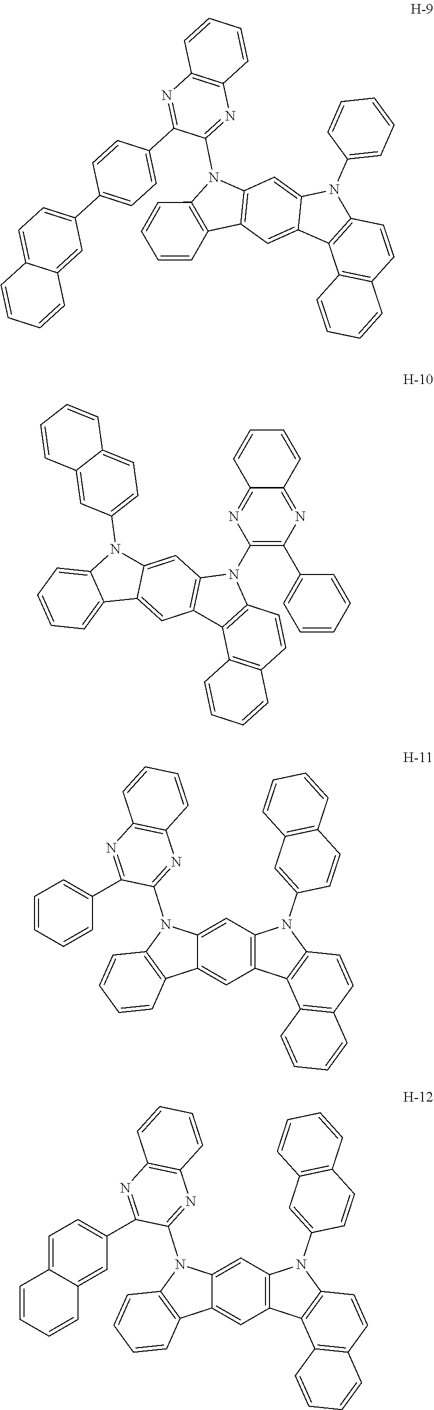

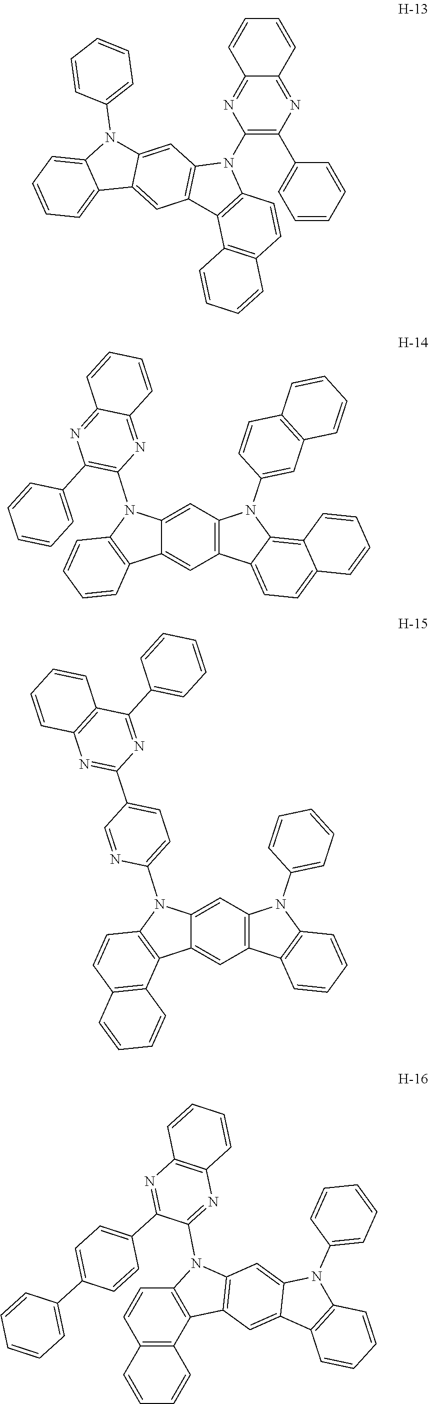 [SMILES: c1ccc(-c2nc3ccccc3nc2-n2c3cc4c(cc3c3c5ccccc5ccc32)c2ccccc2n4-c2ccccc2)cc1 ; c1ccc(-c2nc3ccccc3nc2-n2c3ccccc3c3cc4c5ccc6ccccc6c5n(-c5ccc6ccccc6c5)c4cc32)cc1 ; c1ccc(-c2nc(-c3ccc(-n4c5cc6c(cc5c5c7ccccc7ccc54)c4ccccc4n6-c4ccccc4)nc3)nc3ccccc23)cc1 ; c1ccc(-c2ccc(-c3nc4ccccc4nc3-n3c4cc5c(cc4c4c6ccccc6ccc43)c3ccccc3n5-c3ccccc3)cc2)cc1]

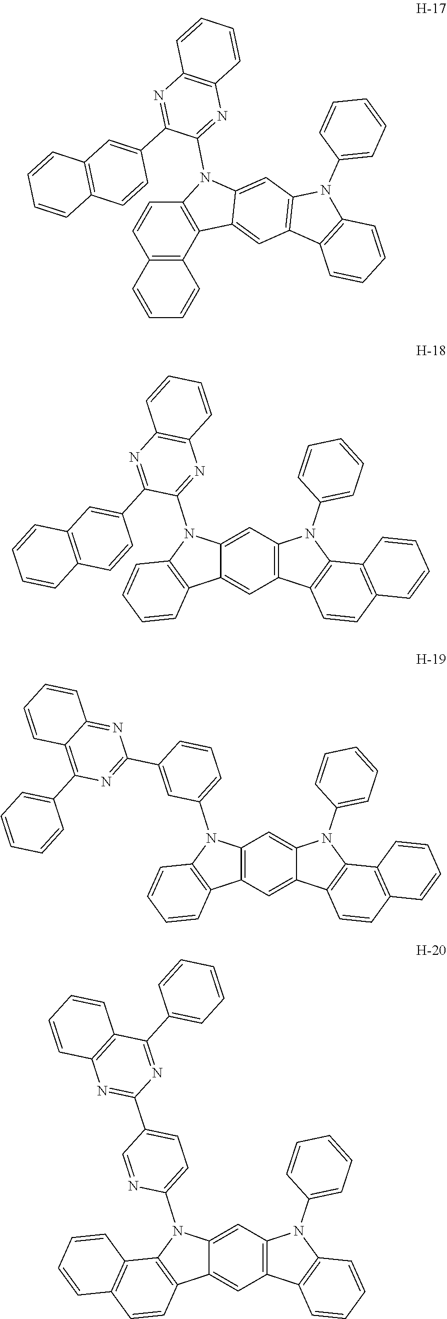

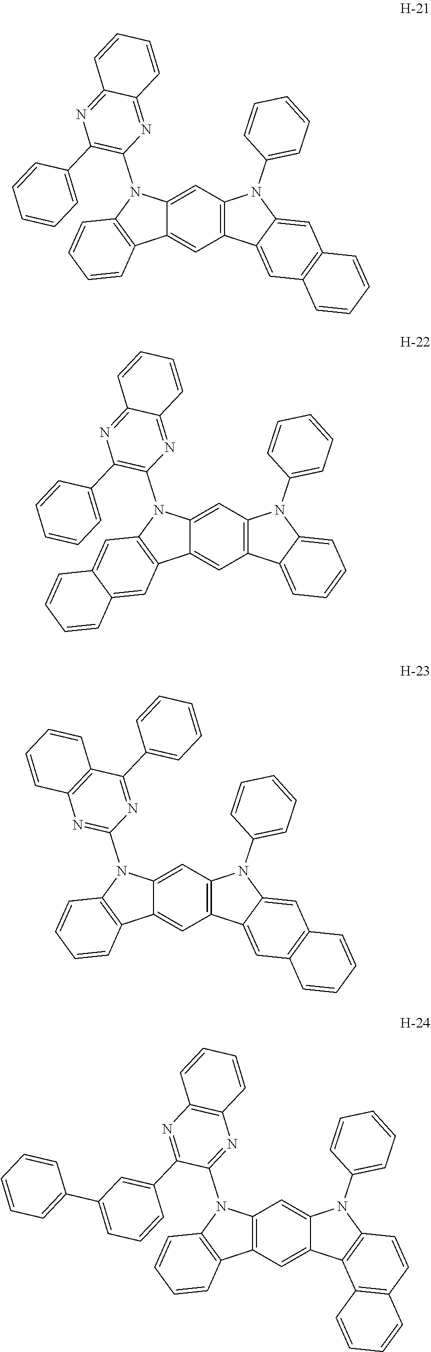 [SMILES: c1ccc(-c2nc3ccccc3nc2-n2c3ccccc3c3cc4c5cc6ccccc6cc5n(-c5ccccc5)c4cc32)cc1 ; c1ccc(-c2nc3ccccc3nc2-n2c3cc4ccccc4cc3c3cc4c5ccccc5n(-c5ccccc5)c4cc32)cc1 ; c1ccc(-c2nc(-n3c4ccccc4c4cc5c6cc7ccccc7cc6n(-c6ccccc6)c5cc43)nc3ccccc23)cc1 ; c1ccc(-c2cccc(-c3nc4ccccc4nc3-n3c4ccccc4c4cc5c6c7ccccc7ccc6n(-c6ccccc6)c5cc43)c2)cc1]

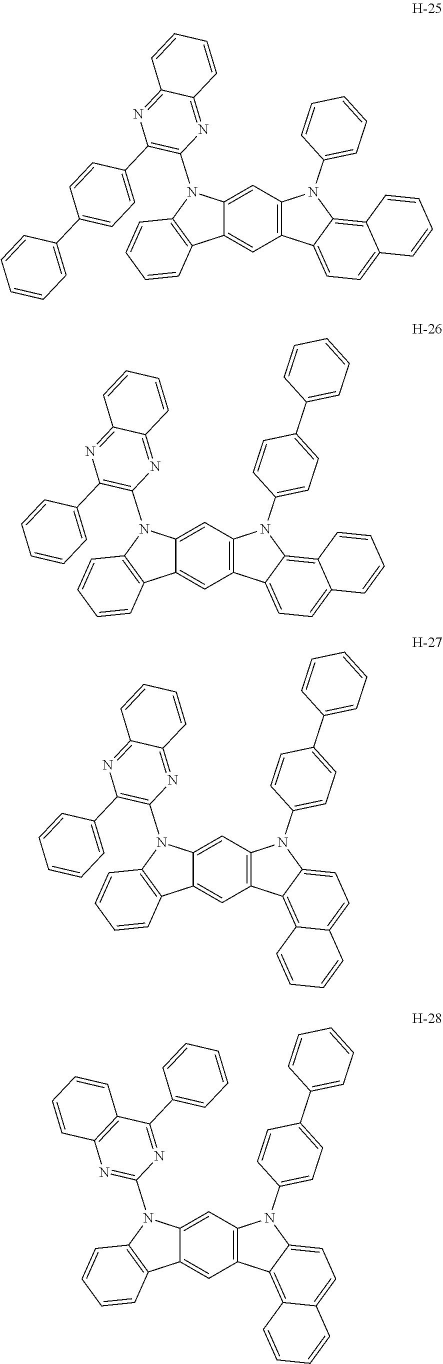

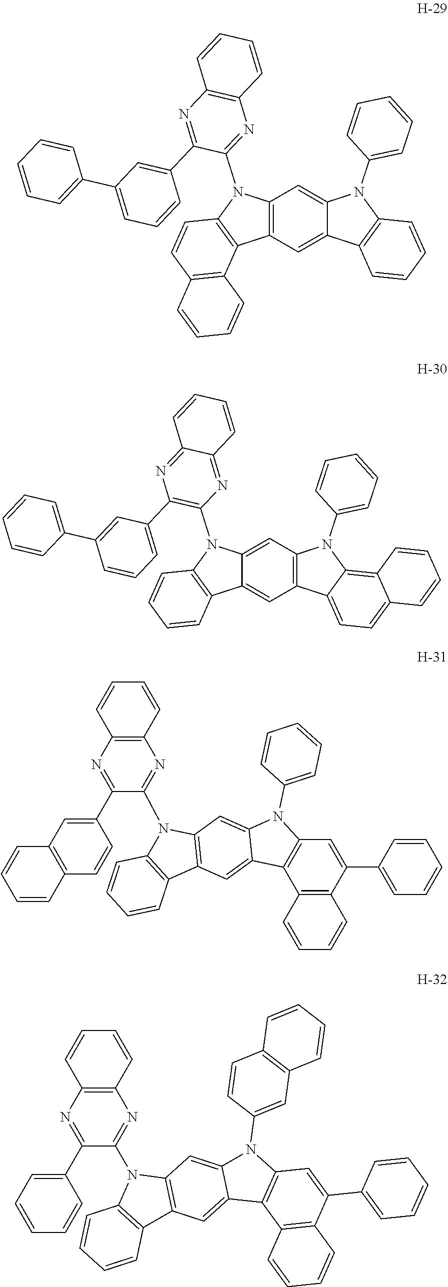 [SMILES: c1ccc(-c2cccc(-c3nc4ccccc4nc3-n3c4cc5c(cc4c4c6ccccc6ccc43)c3ccccc3n5-c3ccccc3)c2)cc1 ; c1ccc(-c2cccc(-c3nc4ccccc4nc3-n3c4ccccc4c4cc5c6ccc7ccccc7c6n(-c6ccccc6)c5cc43)c2)cc1 ; c1ccc(-c2cc3c(c4ccccc24)c2cc4c5ccccc5n(-c5nc6ccccc6nc5-c5ccc6ccccc6c5)c4cc2n3-c2ccccc2)cc1 ; c1ccc(-c2nc3ccccc3nc2-n2c3ccccc3c3cc4c5c6ccccc6c(-c6ccccc6)cc5n(-c5ccc6ccccc6c5)c4cc32)cc1]

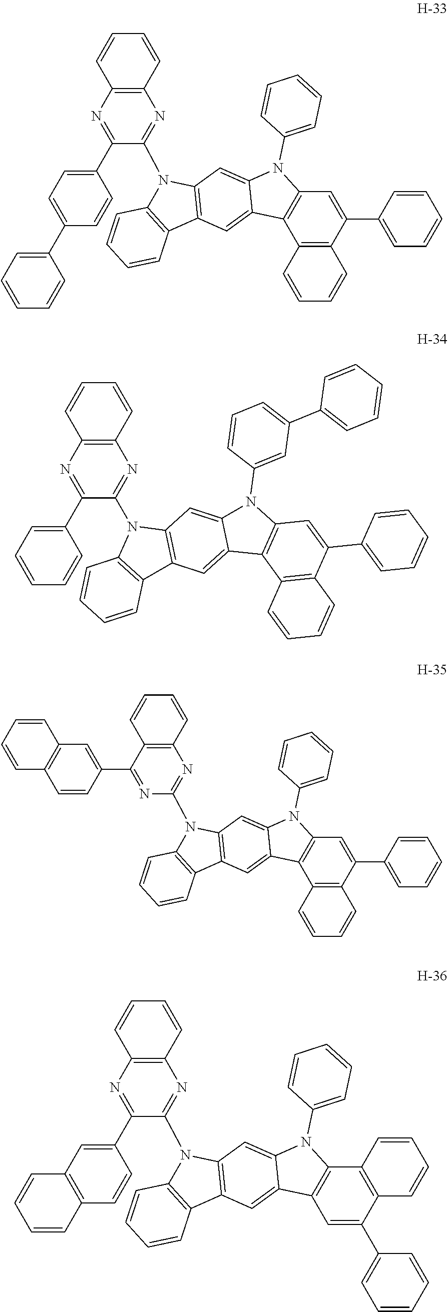 [SMILES: c1ccc(-c2ccc(-c3nc4ccccc4nc3-n3c4ccccc4c4cc5c6c7ccccc7c(-c7ccccc7)cc6n(-c6ccccc6)c5cc43)cc2)cc1 ; c1ccc(-c2cccc(-n3c4cc5c(cc4c4c6ccccc6c(-c6ccccc6)cc43)c3ccccc3n5-c3nc4ccccc4nc3-c3ccccc3)c2)cc1 ; c1ccc(-c2cc3c(c4ccccc24)c2cc4c5ccccc5n(-c5nc(-c6ccc7ccccc7c6)c6ccccc6n5)c4cc2n3-c2ccccc2)cc1 ; c1ccc(-c2cc3c4cc5c6ccccc6n(-c6nc7ccccc7nc6-c6ccc7ccccc7c6)c5cc4n(-c4ccccc4)c3c3ccccc23)cc1]

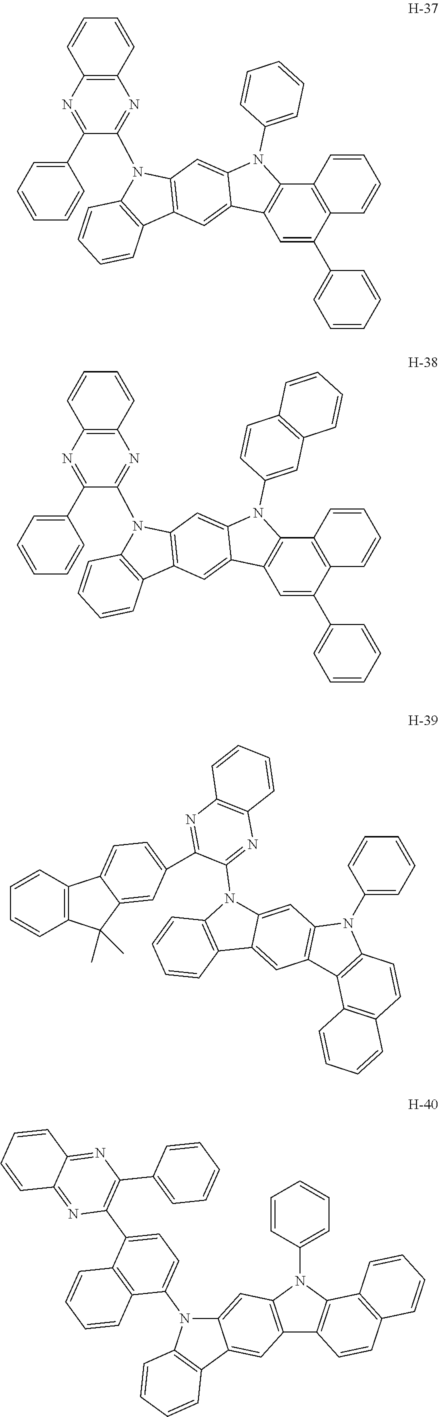

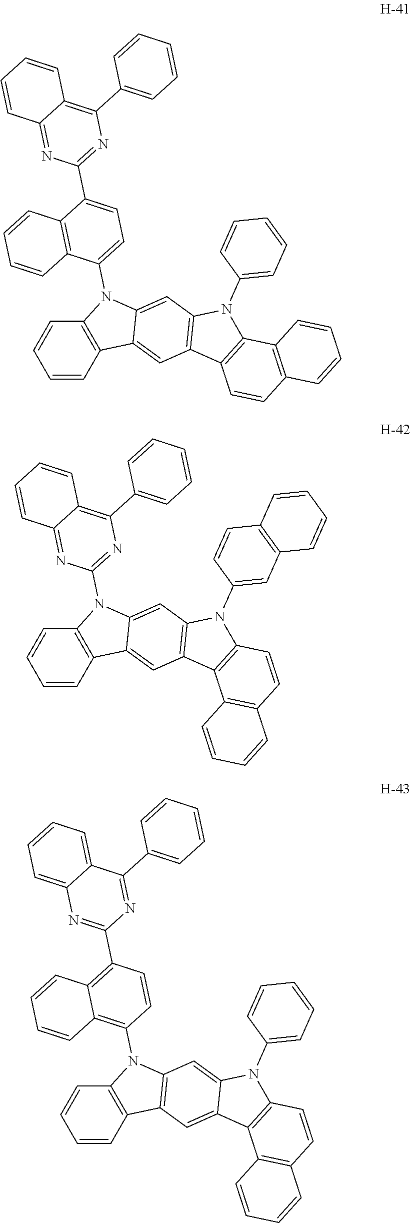 [SMILES: c1ccc(-c2nc(-c3ccc(-n4c5ccccc5c5cc6c7ccc8ccccc8c7n(-c7ccccc7)c6cc54)c4ccccc34)nc3ccccc23)cc1 ; c1ccc(-c2nc(-n3c4ccccc4c4cc5c6c7ccccc7ccc6n(-c6ccc7ccccc7c6)c5cc43)nc3ccccc23)cc1 ; c1ccc(-c2nc(-c3ccc(-n4c5ccccc5c5cc6c7c8ccccc8ccc7n(-c7ccccc7)c6cc54)c4ccccc34)nc3ccccc23)cc1]

The organic electroluminescent compounds represented by formulas 1 and 2 of the present disclosure may be produced by a synthetic method known to one skilled in the art. For example, the compound represented by formula 1 may be synthesized as shown in the following reaction scheme 1, and the compound represented by formula 2 may be synthesized referring to the method of the following reaction scheme 2 or 3, but is not limited thereto.

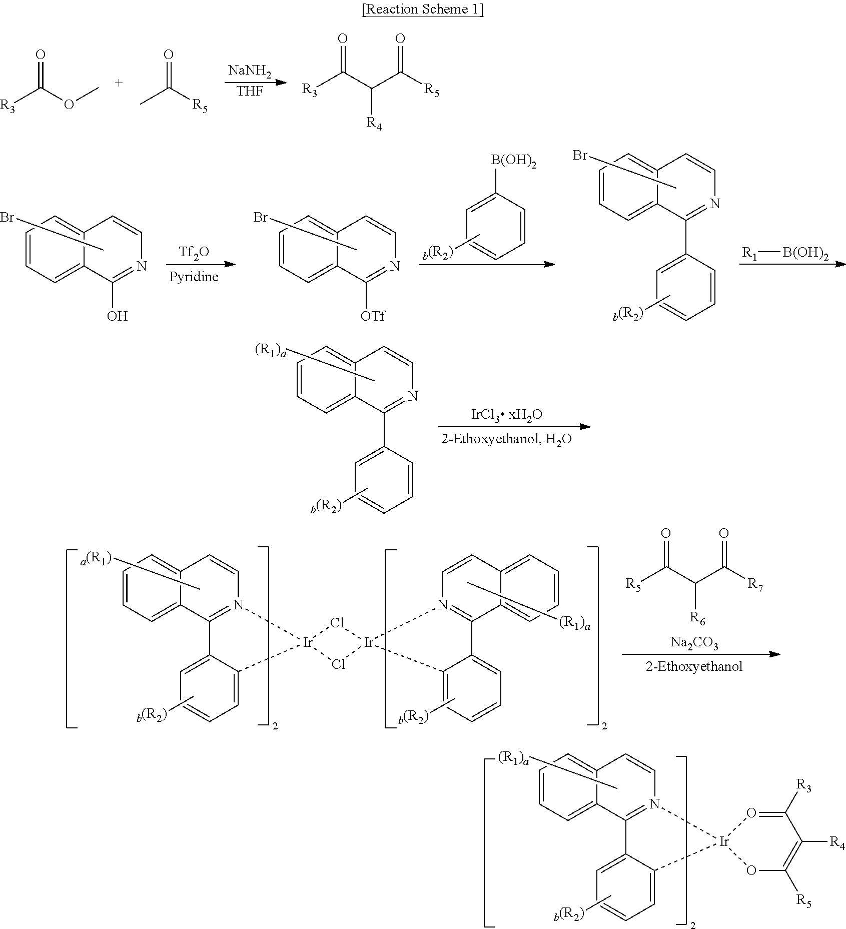

In reaction scheme 1, R₁ to R₅, a, and b are as defined in formula 1.

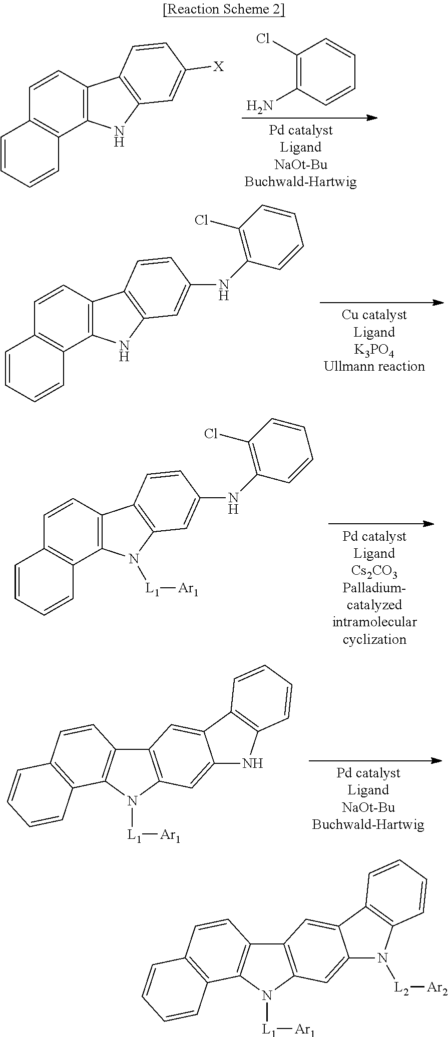

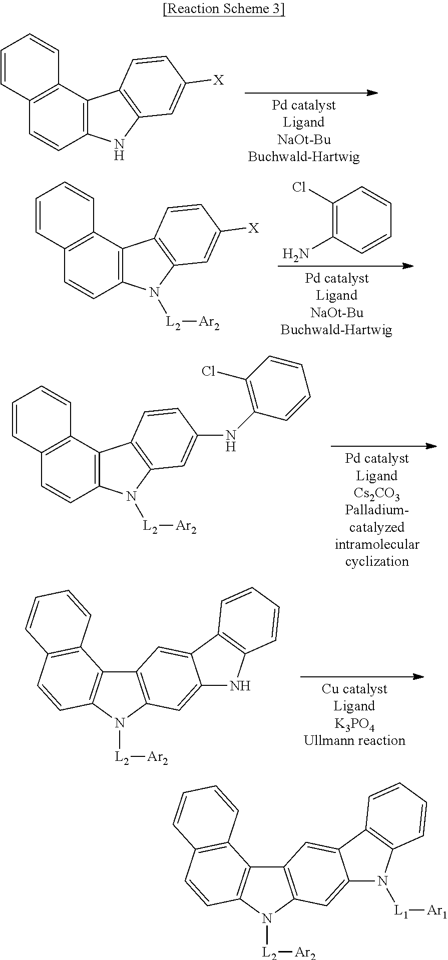

In reaction schemes 2 and 3, Ar₁, Ar₂, L₁, and L₂ are as defined in formula 2.

Meanwhile, the organic electroluminescent device of the present disclosure may comprise a first electrode, a second electrode, and at least one organic layer between the first and second electrodes. One of the first and second electrodes may be an anode, and the other may be a cathode. The organic layer may comprise at least one light-emitting layer, and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. The light-emitting layer is a layer from which light is emitted, and can be a single layer or a multi-layer of which two or more layers are stacked. In the light-emitting layer, it is preferable that the doping concentration of the dopant compound based on the host compound is less than 20 wt %.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. Also, the hole auxiliary layer may be placed between the hole transport layer (or hole injection layer) and the light-emitting layer, and may be effective to promote or block the hole transport rate (or the hole injection rate), thereby enabling the charge balance to be controlled. In addition, the electron blocking layer may be placed between the hole transport layer (or hole injection layer) and the light-emitting layer, and may block overflowing electrons from the light-emitting layer and confine the excitons in the light-emitting layer to prevent light leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifespan of the organic electroluminescent device.

According to one aspect of the present disclosure, the organic layer comprising a combination of at least one compound represented by formula 1 and at least one compound represented by formula 2 may be provided. The organic layer may comprise a plurality of layers, and the compound represented by formula 1 and the compound represented by formula 2 may be comprised in the same layer or different layers, respectively. Also, the present disclosure provides an organic electroluminescent device comprising the organic layer.

According to another aspect of the present disclosure, the combination of the dopant and the host, which is a combination of at least one dopant compound represented by the formula 1 and at least one host compound represented by the formula 2, may be provided. Also, the present disclosure provides an organic electroluminescent device comprising the combination of the dopant and the host.

According to a further aspect of the present disclosure, an organic electroluminescent material comprising a combination of at least one compound represented by formula 1 and at least one compound represented by formula 2, and an organic electroluminescent device comprising the material may be provided. The material may consist of only the combination of the compound of formula 1 and the compound of formula 2, and may further comprise conventional materials comprised in an organic electroluminescent material.

Also, the organic electroluminescent device of the present disclosure may comprise the compounds of formulas 1 and 2, and further comprise at least one compound selected from the group consisting of arylamine-based compounds and styrylarylamine-based compounds.

In addition, in the organic electroluminescent device of the present disclosure, the organic layer may further comprise at least one metal selected from the group consisting of metals of Group 1, metals of Group 2, transition metals of the 4^{th} period, transition metals of the 5^{th} period, lanthanides and organic metals of d-transition elements of the Periodic Table, or at least one complex compound comprising said metal, besides the compounds of formulas 1 and 2. Further, the organic layer may further comprise a light-emitting layer and a charge generating layer.

Further, the organic electroluminescent device of the present disclosure may emit white light by further including at least one light-emitting layer containing a blue, red or green light-emitting compound, which are known in the art. Also, it may further comprise a yellow or orange light-emitting layer, if necessary.

In the organic electroluminescent device of the present disclosure, at least one layer selected from a chalcogenide layer, a metal halide layer and a metal oxide layer (hereinafter, “a surface layer”) may be preferably placed on an inner surface(s) of one or both electrodes. Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer may provide operation stability for the organic electroluminescent device. Preferably, the chalcogenide includes SiO_{X}(1≤X≤2), AlO_{X}(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In the organic electroluminescent device of the present disclosure, a mixed region of an electron transport compound and a reductive dopant, or a mixed region of a hole transport compound and an oxidative dopant is preferably placed on at least one surface of a pair of electrodes. In this case, the electron transport compound is reduced to an anion, and thus it becomes easier to inject and transport electrons from the mixed region to an electroluminescent medium. Further, the hole transport compound is oxidized to a cation, and thus it becomes easier to inject and transport holes from the mixed region to the electroluminescent medium. Preferably, the oxidative dopant includes various Lewis acids and acceptor compounds; and the reductive dopant includes alkali metals, alkali metal compounds, alkaline earth metals, rare-earth metals, and mixtures thereof. A reductive dopant layer may be employed as a charge-generating layer to prepare an organic electroluminescent device having two or more light-emitting layers and emitting white light.

In order to form each layer of the organic electroluminescent device of the present disclosure, dry film-forming methods such as vacuum evaporation, sputtering, plasma and ion plating methods, or wet film-forming methods such as ink jet printing, nozzle printing, slot coating, spin coating, dip coating, and flow coating methods may be used. The dopant and host compounds of the present disclosure may be co-evaporated or mixture-evaporated.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

The co-evaporation is a mixed deposition method in which two or more isomer materials are placed in a respective individual crucible source and a current is applied to both cells at the same time to evaporate the materials. The mixture-evaporation is a mixed deposition method in which two or more isomer materials are mixed in one crucible source before evaporating them, and a current is applied to the cell to evaporate the materials.

A display system or a lighting system can be produced by using the organic electroluminescent device of the present disclosure.

Hereinafter, the luminous properties of the organic light-emitting diode (OLED) device comprising the dopant compound and the host compound of the present disclosure will be explained in detail by comparing with the conventional OLED device. However, the present disclosure is not limited by the following examples.

### Device Example 1: Producing an OLED Device Comprising the Compound of the Present Disclosure

An OLED device was produced by using the organic electroluminescent compound according to the present disclosure. A transparent electrode indium tin oxide (ITO) thin film (10 Ω/sq) on a glass substrate for an OLED device (GEOMATEC CO., LTD., Japan) was subjected to an ultrasonic washing with acetone, ethanol, and distilled water, sequentially, and then was stored in isopropanol. The ITO substrate was then mounted on a substrate holder of a vacuum vapor deposition apparatus. Compound HI-1 was introduced into a cell of the vacuum vapor deposition apparatus, and then the pressure in the chamber of the apparatus was controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer.

Compound HT-1 was then introduced into another cell of the vacuum vapor deposition apparatus and was evaporated by applying an electric current to the cell, thereby forming a first hole transport layer having a thickness of 10 nm on the second hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layer, a light-emitting layer was formed thereon as follows: Compound H-2 was introduced into one cell of the vacuum vapor depositing apparatus as a host, and compound D-19 was introduced into another cell as a dopant. The dopant was deposited in a doping amount of 3 wt % based on the total amount of the host and dopant to form a light-emitting layer having a thickness of 40 nm on the second hole transport layer. Compound ET-1 and compound EI-1 were then introduced into the other two cells and evaporated at a rate of 1:1 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED device was produced.

### Device Example 2: Producing an OLED Device Comprising the Compound of the Present Disclosure

An OLED device was produced in the same manner as in Device Example 1, except that compound HT-3 was used as a second hole transport layer instead of compound HT-2, and compound D-7 was used as a dopant.

### Device Example 3: Producing an OLED Device Comprising the Compound of the Present Disclosure

An OLED device was produced in the same manner as in Device Example 1, except that compound HI-1 was deposited as a first hole injection layer having a thickness of 90 nm; compound HT-3 was used as a second hole transport layer instead of compound HT-2; compound D-9 was used as a dopant; and the dopant was deposited in a doping amount of 2 wt % based on the total amount of the host and the dopant.

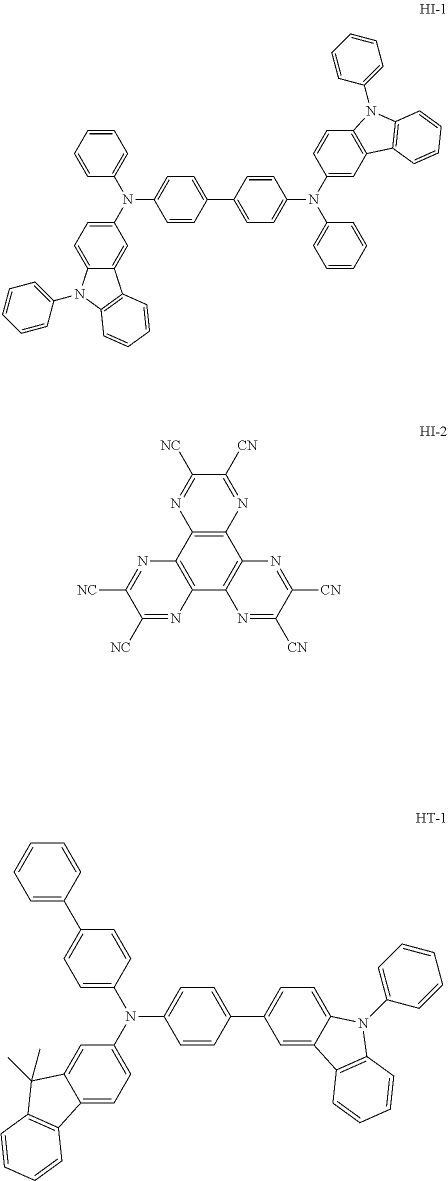 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21]

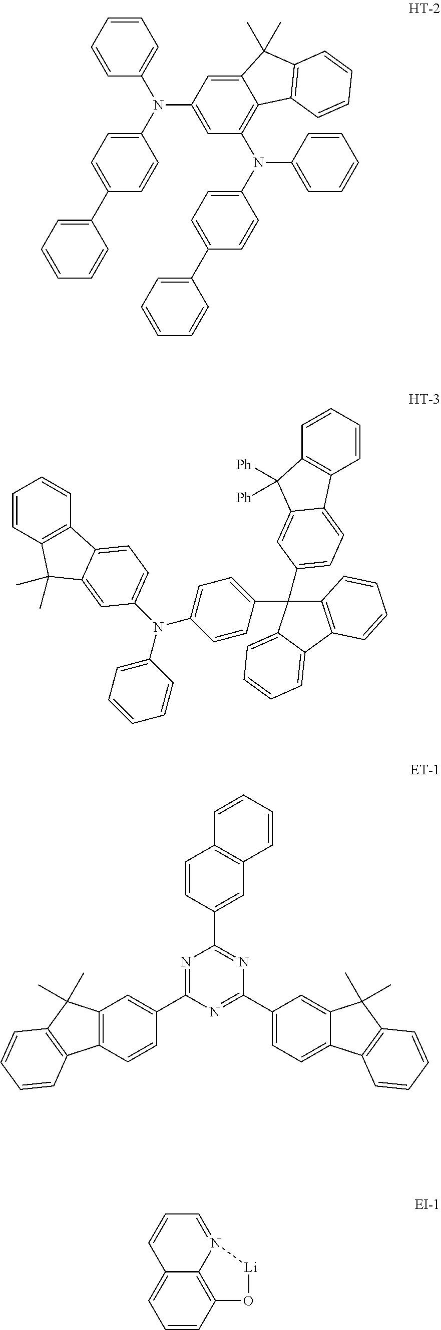

### Comparative Example 1: Producing an OLED Device Comprising a Conventional Compound

An OLED device was produced in the same manner as in Device Example 1, except that the following compound A was used as a dopant.

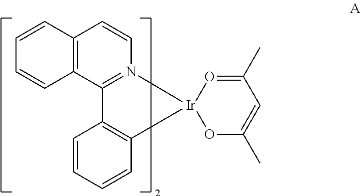

### Comparative Example 2: Producing an OLED Device Comprising a Conventional Compound

An OLED device was produced in the same manner as in Device Example 1, except that the following compound B was used as a dopant.

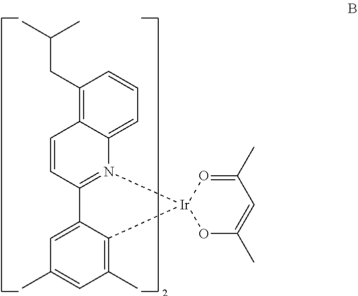

### Comparative Example 3: Producing an OLED Device Comprising a Conventional Compound

An OLED device was produced in the same manner as in Device Example 1, except that compound B above was used as a dopant, and compound HT-3 was used as a second hole transport layer instead of compound HT-2.

Comparison of Color Reproduction Range

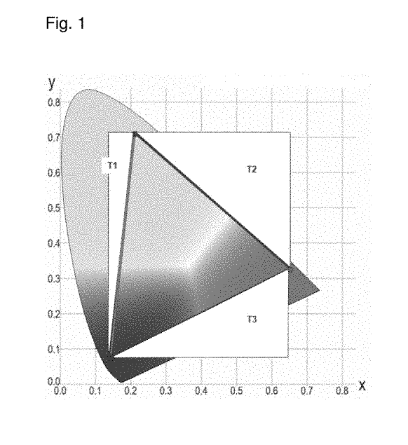

The color reproduction range was calculated under the standard of the color space made by the National Television System Committee (NTSC), which is based on the color coordinate system defined by the International Commission on Illumination (CIE). Referring to FIG. 1, the area of the triangle formed by the three points of red (0.67, 0.33), green (0.21, 0.71), and blue (0.14, 0.08) defined by NTSC is calculated (hereinafter, “NTSC area”). Also, the area of the triangle is calculated by using NTSC definition values for blue and green, and the measured values in the produced device for red, and the ratio of the triangle area versus the NTSC area is then calculated. The higher the figure, the more vivid color is reproduced.

The percentages (%) of the areas of the Comparative Examples and the Device Examples versus the NTSC area, i.e. NTSC Color Space, are calculated as shown in Table 1 below.

TABLE 1

| | |
|---|---|
| Whole | {R(x) − B(x)} * {G(y) − B(y)} |
| Rectangle | |
| Triangle 1 (T1) | {G(x) − B(x)} * {G(y) − B(y)}/2 |
| Triangle 2 (T2) | {R(x) − G(x)} * {G(y) − R(y)}/2 |
| Triangle 3 (T3) | {R(x) − B(x)} * {R(y) − B(y)}/2 |
| NTSC Color | Whole Rectangle − (Triangle 1 + Triangle 2 + |
| Space | Triangle 3) |

In Table 1, R(x) represents CIE X coordinate of red light-emission, R(y) represents CIE Y coordinate of red light-emission, G(x) represents CIE X coordinate of green light-emission, G(y) represents a CIE Y coordinate of green light-emission, B(x) represents a CIE X coordinate of blue light-emission, and B(y) represents a CIE Y coordinate of blue light-emission.

The CIE color coordinates of the organic electroluminescent devices of the Device Examples and the Comparative Examples, and the percentages of the area versus the NTSC area are shown in Table 2 below.

TABLE 2

| | Device | Device | Device | Comparative | Comparative | Comparative |
|---|---|---|---|---|---|---|
| | Example 1 | Example 2 | Example 3 | Example 1 | Example 2 | Example 3 |
| CIE X | 0.693 | 0.700 | 0.697 | 0.677 | 0.664 | 0.664 |
| CIE Y | 0.307 | 0.330 | 0.303 | 0.323 | 0.336 | 0.336 |
| NTSC | 105.1% | 106.0% | 106.0% | 101.5% | 98.7% | 98.7% |
| Color | | | | | | |
| Space | | | | | | |

From Table 2 above, it can be confirmed that the organic electroluminescent devices comprising the compound of the present disclosure (Device Examples 1 to 3) are superior to the organic electroluminescent devices comprising the conventional compound (Comparative Examples 1 to 3) in color reproduction range (color gamut).

1. An organic electroluminescent material comprising at least one compound represented by the following formula 1: 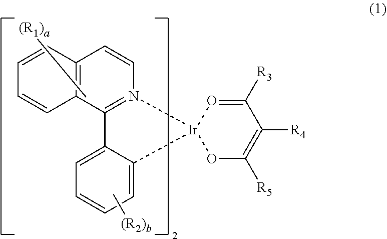
wherein
R₁ and R₂, each independently, represent hydrogen, deuterium, a substituted or unsubstituted (C1-C10)alkyl, a substituted or unsubstituted (C2-C10)alkenyl, or a substituted or unsubstituted (C6-C30)aryl, with a proviso that both R₁ and R₂ are not hydrogen,
R₃ to R₅, each independently, represent hydrogen, deuterium, a substituted or unsubstituted (C1-C10)alkyl, or a substituted or unsubstituted (C6-C30)aryl, and
a represents an integer of 1 to 6, and b represents an integer of 1 to 4, where if a and b, each independently, are an integer of 2 or more, each of R₁ and R₂ may be the same or different; and
at least one compound represented by the following formula 2: 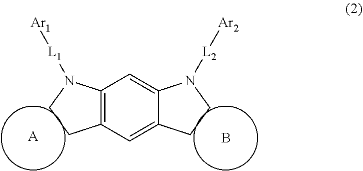
wherein
A ring and B ring, each independently, represent a substituted or unsubstituted benzene ring, or a substituted or unsubstituted naphthalene ring, with a proviso that at least one of A ring and B ring is a substituted or unsubstituted naphthalene ring,
Ar₁ and Ar₂, each independently, represent a substituted or unsubstituted (C6-C30)aryl, or a substituted or unsubstituted nitrogen-containing (8- to 30-membered)heteroaryl,
L₁ and L₂, each independently, represent a single bond, a substituted or unsubstituted (C6-C30)arylene, or a substituted or unsubstituted (3- to 30-membered)heteroarylene, and
the heteroaryl(ene) contains at least one heteroatom selected from B, N, O, S, Si, and P.

2. The organic electroluminescent material according to claim 1,
wherein
R₁ represents hydrogen, or a substituted or unsubstituted (C1-C6)alkyl,
R₂ represents a substituted or unsubstituted (C1-C6)alkyl,
R₃ and R₅, each independently, represent a substituted or unsubstituted (C1-C6)alkyl, or a substituted or unsubstituted (C6-C18)aryl,
R₄ represents hydrogen, a substituted or unsubstituted (C1-C6)alkyl, or a substituted or unsubstituted (C6-C18)aryl,
a and b, each independently, represent 1 or 2, where if a and b, each independently, are 2, each of R₁ and R₂ may be the same or different.

3. The organic electroluminescent material according to claim 1, wherein formula 2 is represented by the following formula 3: 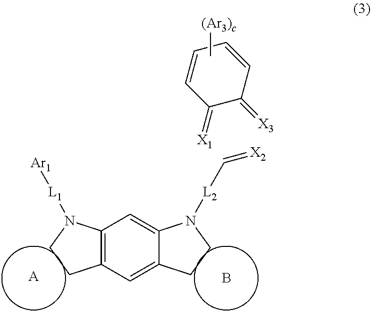
wherein
any one of A ring and B ring represents a substituted or unsubstituted naphthalene ring, and the other represents a substituted or unsubstituted benzene ring,
X₁ to X₃, each independently, represent CR₆ or N, with a proviso that at least one of X₁ to X₃ represents N,
R₆ represents hydrogen, or a substituted or unsubstituted (C6-C30)aryl,
Ar₃ represents hydrogen, a substituted or unsubstituted (C1-C30)alkyl, a substituted or unsubstituted (C6-C30)aryl, or a substituted or unsubstituted (3- to 30-membered)heteroaryl,
c represents an integer of 1 to 4, where if c is an integer of 2 or more, each of Ar₃ may be the same or different, and
Ar₁, L₁, and L₂ are as defined in claim 1.

4. The organic electroluminescent material according to claim 1, wherein the substituents of the substituted alkyl, the substituted alkenyl, the substituted aryl(ene), the substituted heteroaryl(ene), the substituted benzene ring, and the substituted naphthalene ring in R₁ to R₅, Ar₁, Ar₂, L₁, L₂, A ring, and B ring, each independently, are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a (3- to 7-membered)heterocycloalkyl, a (C6-C30)aryloxy, a (C6-C30)arylthio, a (C6-C30)aryl, a (5- to 30-membered)heteroaryl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

5. The organic electroluminescent material according to claim 1, wherein the compound represented by formula 1 is selected from the group consisting of:

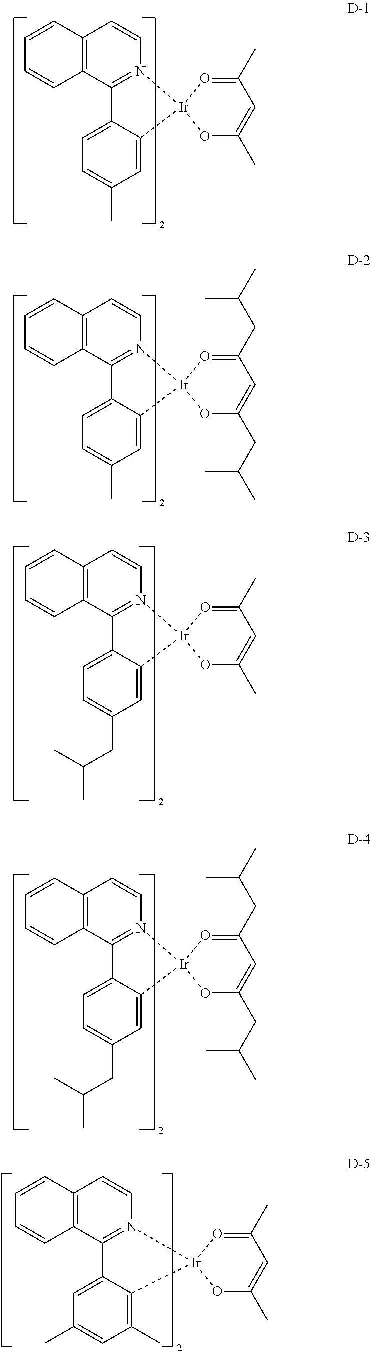

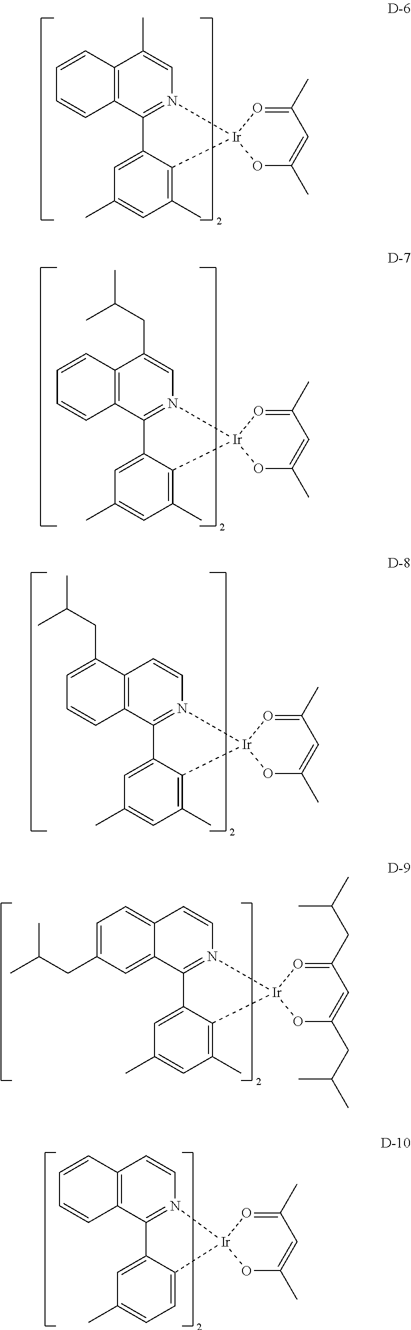

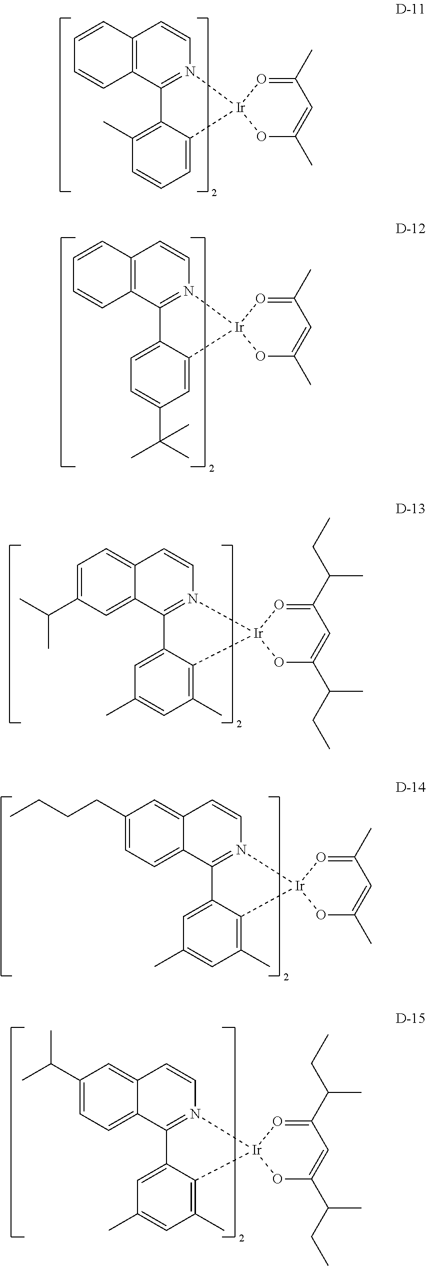

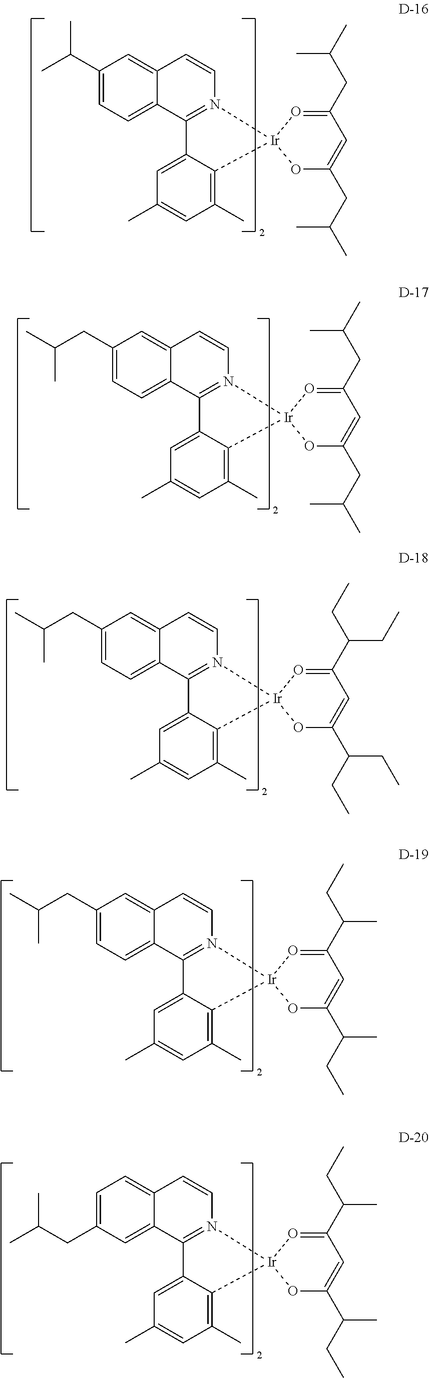

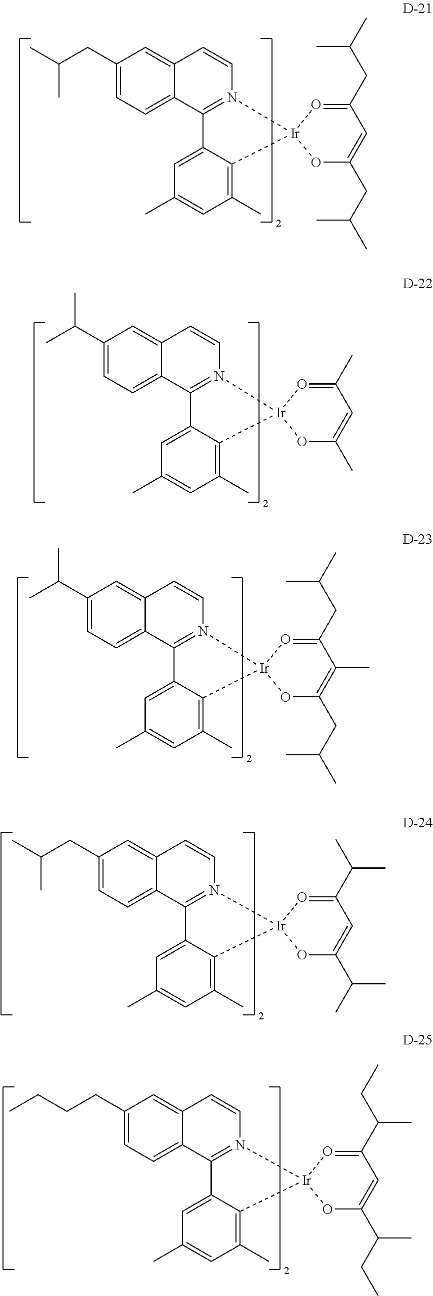

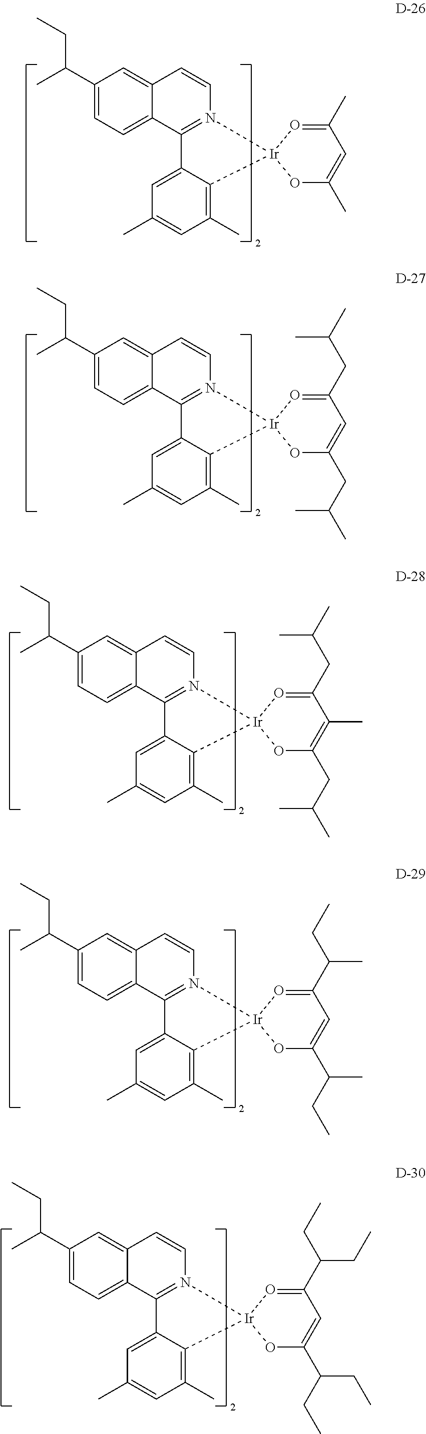

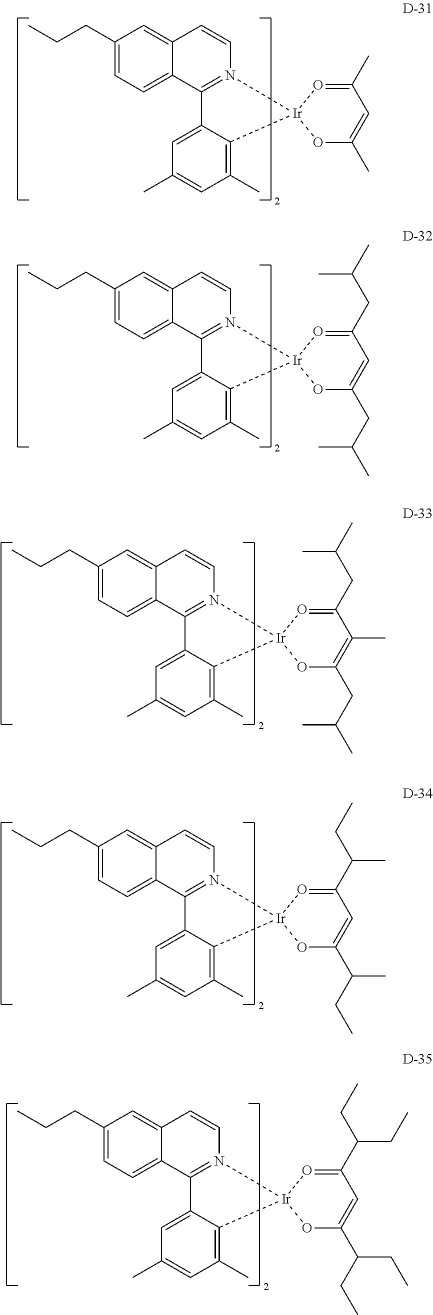

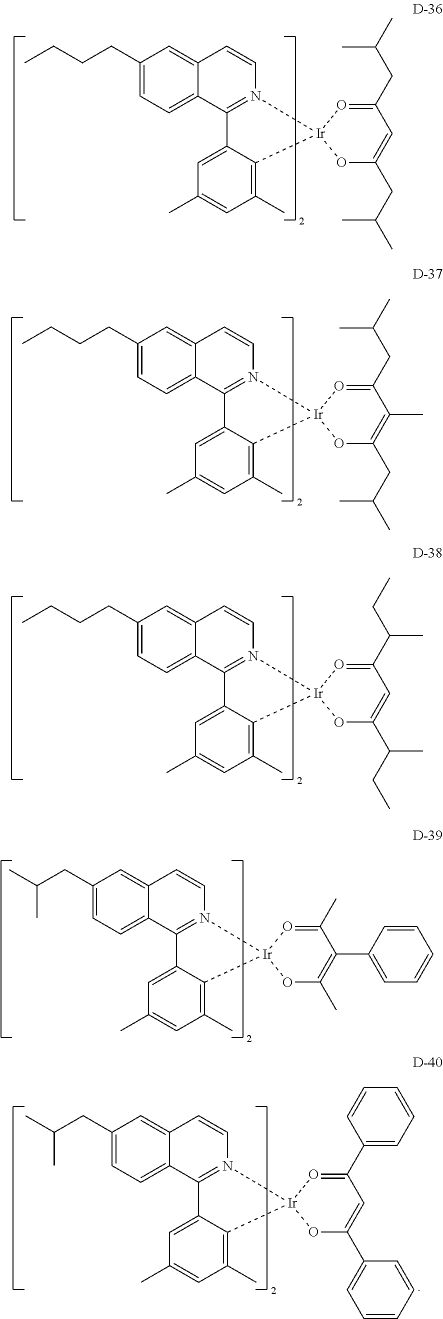

6. The organic electroluminescent material according to claim 1, wherein the compound represented by formula 2 is selected from the group consisting of:

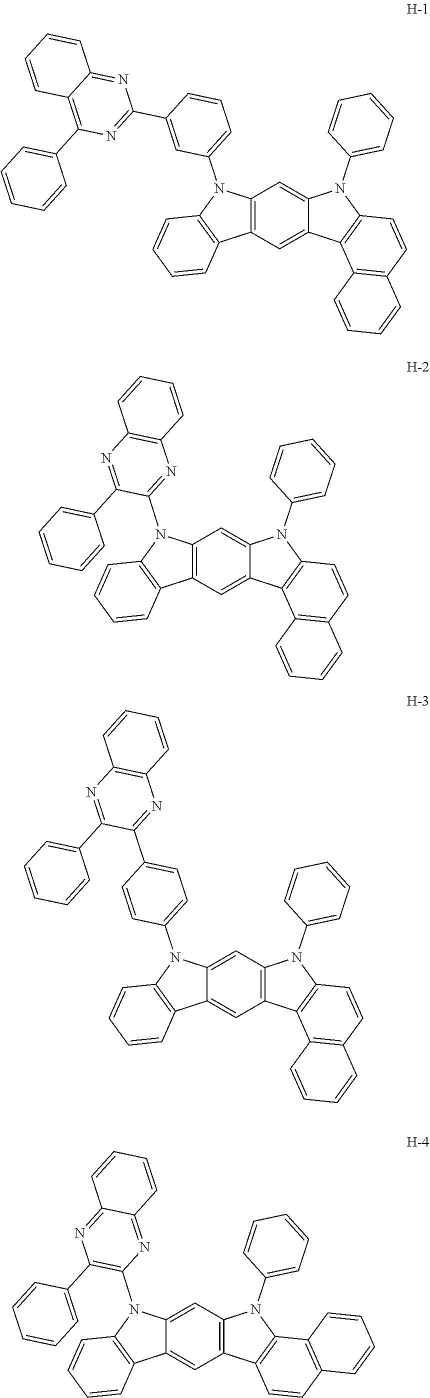 [SMILES: c1ccc(-c2nc(-c3cccc(-n4c5ccccc5c5cc6c7c8ccccc8ccc7n(-c7ccccc7)c6cc54)c3)nc3ccccc23)cc1 ; c1ccc(-c2nc3ccccc3nc2-n2c3ccccc3c3cc4c5c6ccccc6ccc5n(-c5ccccc5)c4cc32)cc1 ; c1ccc(-c2nc3ccccc3nc2-c2ccc(-n3c4ccccc4c4cc5c6c7ccccc7ccc6n(-c6ccccc6)c5cc43)cc2)cc1 ; c1ccc(-c2nc3ccccc3nc2-n2c3ccccc3c3cc4c5ccc6ccccc6c5n(-c5ccccc5)c4cc32)cc1]

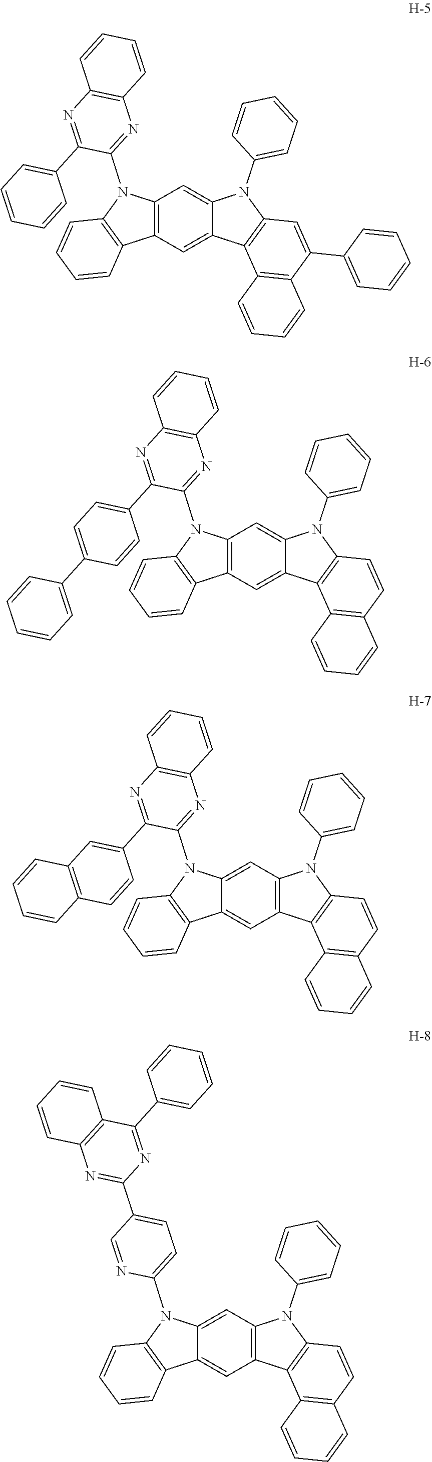 [SMILES: c1ccc(-c2nc3ccccc3nc2-n2c3ccccc3c3cc4c5c6ccccc6c(-c6ccccc6)cc5n(-c5ccccc5)c4cc32)cc1 ; c1ccc(-c2ccc(-c3nc4ccccc4nc3-n3c4ccccc4c4cc5c6c7ccccc7ccc6n(-c6ccccc6)c5cc43)cc2)cc1 ; c1ccc(-n2c3cc4c(cc3c3c5ccccc5ccc32)c2ccccc2n4-c2nc3ccccc3nc2-c2ccc3ccccc3c2)cc1 ; c1ccc(-c2nc(-c3ccc(-n4c5ccccc5c5cc6c7c8ccccc8ccc7n(-c7ccccc7)c6cc54)nc3)nc3ccccc23)cc1]

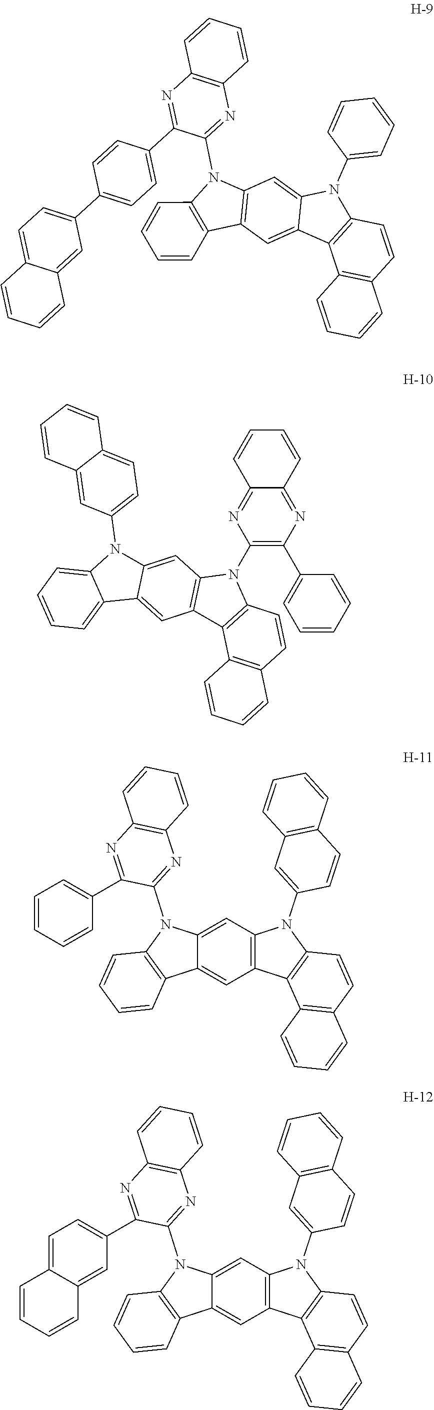

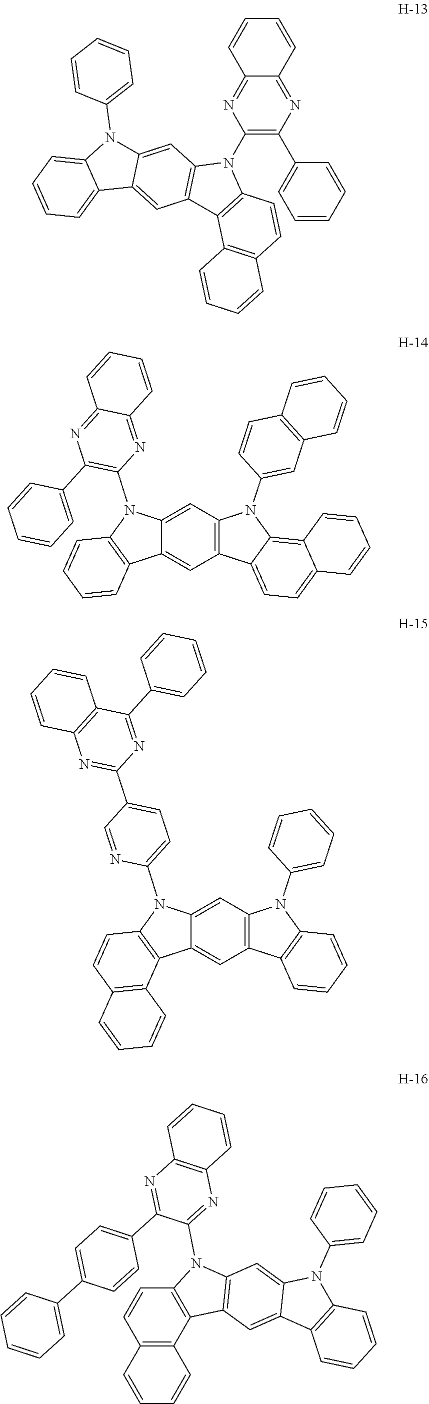 [SMILES: c1ccc(-c2nc3ccccc3nc2-n2c3cc4c(cc3c3c5ccccc5ccc32)c2ccccc2n4-c2ccccc2)cc1 ; c1ccc(-c2nc3ccccc3nc2-n2c3ccccc3c3cc4c5ccc6ccccc6c5n(-c5ccc6ccccc6c5)c4cc32)cc1 ; c1ccc(-c2nc(-c3ccc(-n4c5cc6c(cc5c5c7ccccc7ccc54)c4ccccc4n6-c4ccccc4)nc3)nc3ccccc23)cc1 ; c1ccc(-c2ccc(-c3nc4ccccc4nc3-n3c4cc5c(cc4c4c6ccccc6ccc43)c3ccccc3n5-c3ccccc3)cc2)cc1]

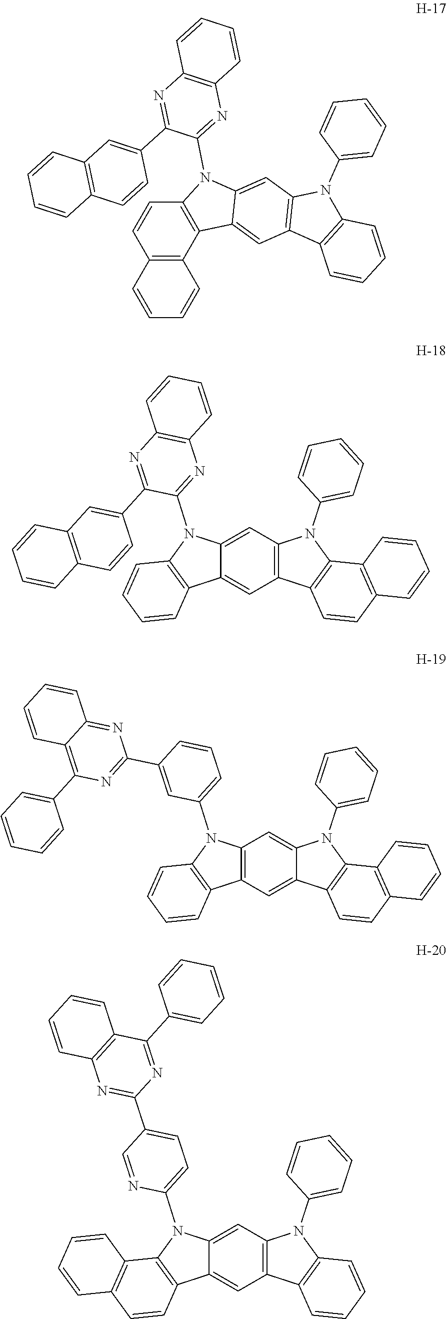

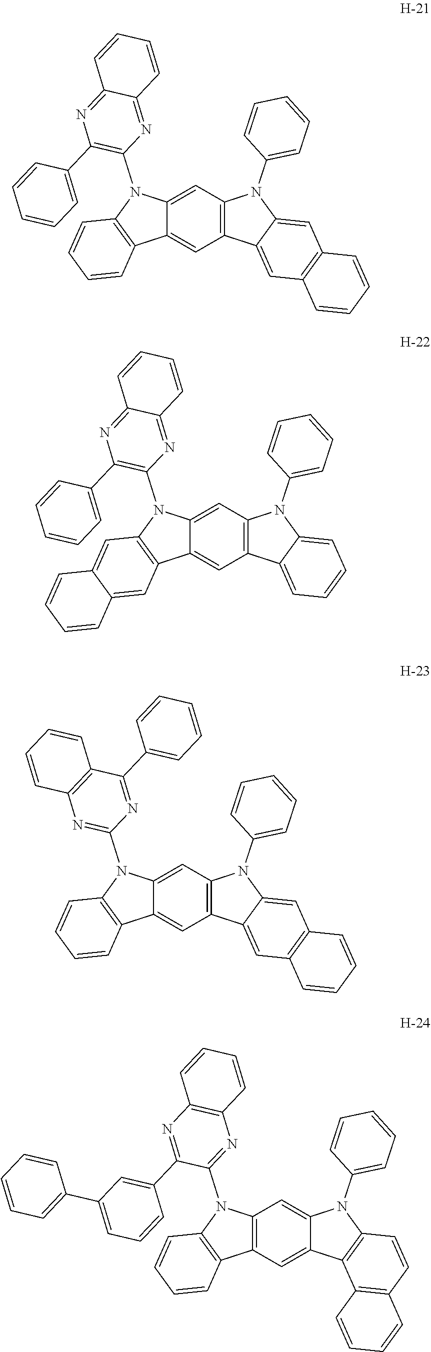 [SMILES: c1ccc(-c2nc3ccccc3nc2-n2c3ccccc3c3cc4c5cc6ccccc6cc5n(-c5ccccc5)c4cc32)cc1 ; c1ccc(-c2nc3ccccc3nc2-n2c3cc4ccccc4cc3c3cc4c5ccccc5n(-c5ccccc5)c4cc32)cc1 ; c1ccc(-c2nc(-n3c4ccccc4c4cc5c6cc7ccccc7cc6n(-c6ccccc6)c5cc43)nc3ccccc23)cc1 ; c1ccc(-c2cccc(-c3nc4ccccc4nc3-n3c4ccccc4c4cc5c6c7ccccc7ccc6n(-c6ccccc6)c5cc43)c2)cc1]

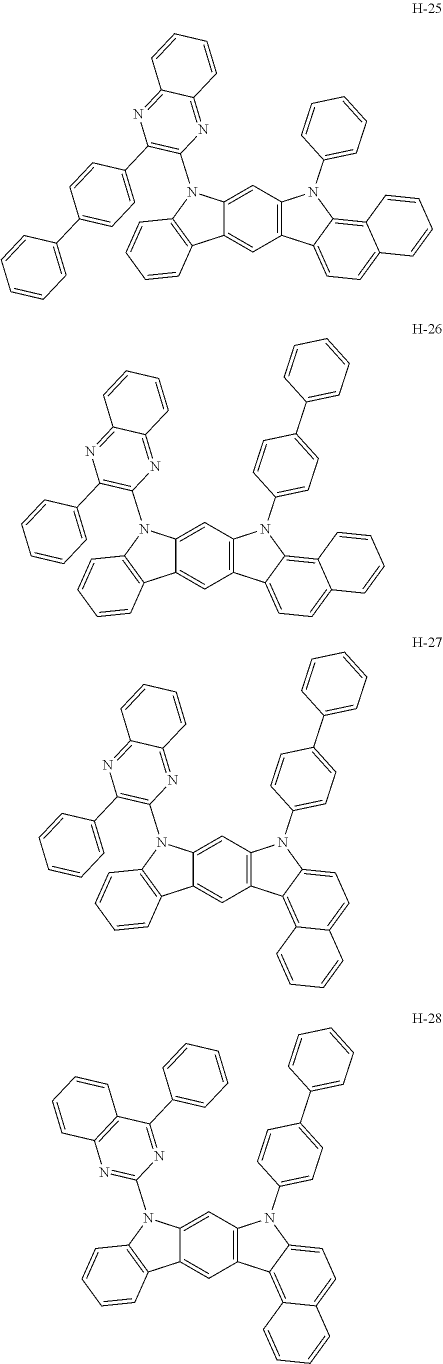

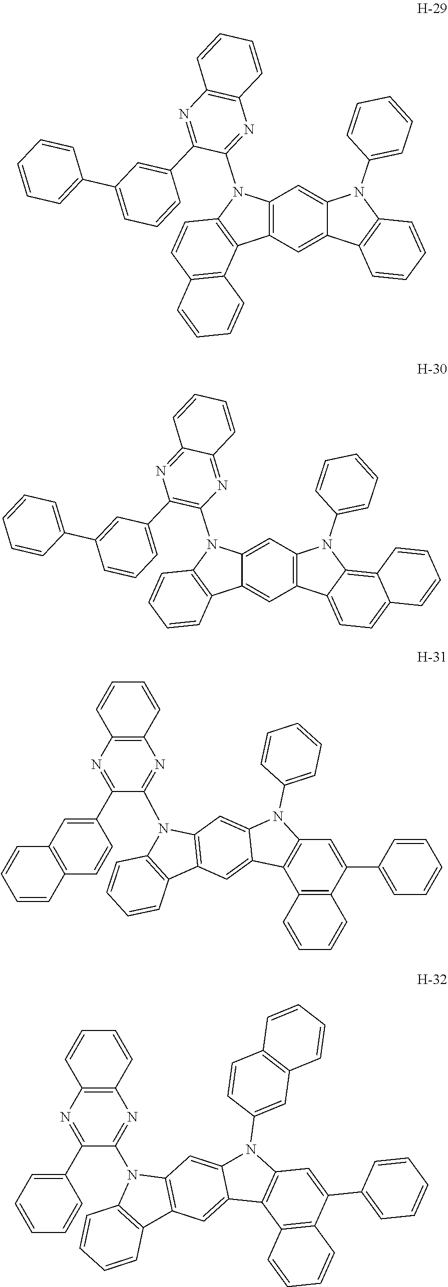 [SMILES: c1ccc(-c2cccc(-c3nc4ccccc4nc3-n3c4cc5c(cc4c4c6ccccc6ccc43)c3ccccc3n5-c3ccccc3)c2)cc1 ; c1ccc(-c2cccc(-c3nc4ccccc4nc3-n3c4ccccc4c4cc5c6ccc7ccccc7c6n(-c6ccccc6)c5cc43)c2)cc1 ; c1ccc(-c2cc3c(c4ccccc24)c2cc4c5ccccc5n(-c5nc6ccccc6nc5-c5ccc6ccccc6c5)c4cc2n3-c2ccccc2)cc1 ; c1ccc(-c2nc3ccccc3nc2-n2c3ccccc3c3cc4c5c6ccccc6c(-c6ccccc6)cc5n(-c5ccc6ccccc6c5)c4cc32)cc1]

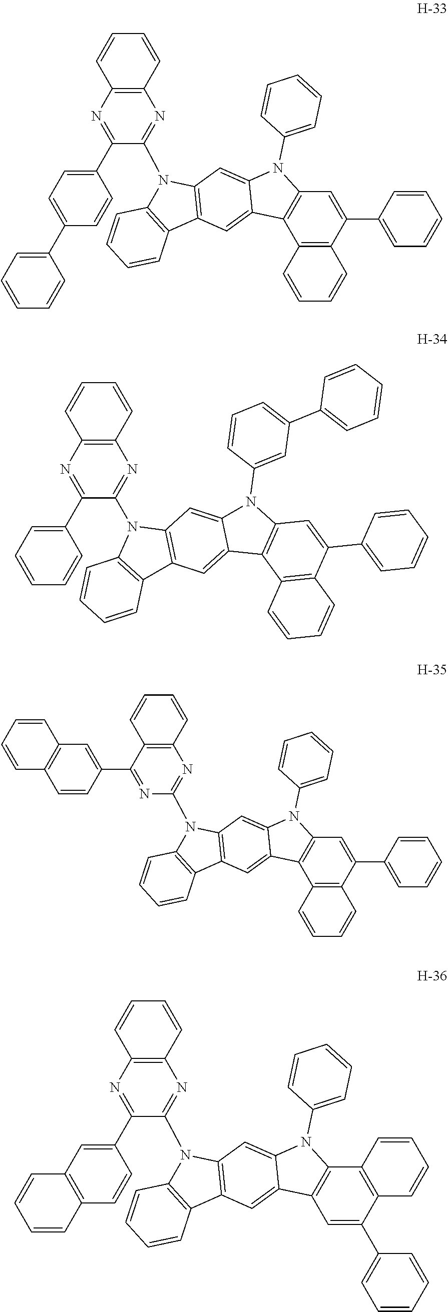 [SMILES: c1ccc(-c2ccc(-c3nc4ccccc4nc3-n3c4ccccc4c4cc5c6c7ccccc7c(-c7ccccc7)cc6n(-c6ccccc6)c5cc43)cc2)cc1 ; c1ccc(-c2cccc(-n3c4cc5c(cc4c4c6ccccc6c(-c6ccccc6)cc43)c3ccccc3n5-c3nc4ccccc4nc3-c3ccccc3)c2)cc1 ; c1ccc(-c2cc3c(c4ccccc24)c2cc4c5ccccc5n(-c5nc(-c6ccc7ccccc7c6)c6ccccc6n5)c4cc2n3-c2ccccc2)cc1 ; c1ccc(-c2cc3c4cc5c6ccccc6n(-c6nc7ccccc7nc6-c6ccc7ccccc7c6)c5cc4n(-c4ccccc4)c3c3ccccc23)cc1]

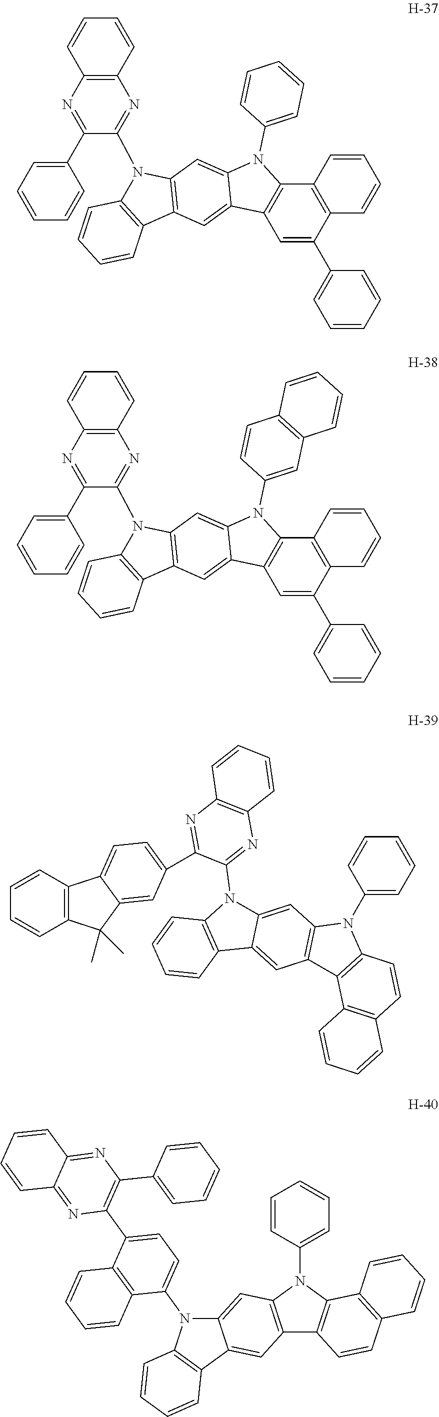

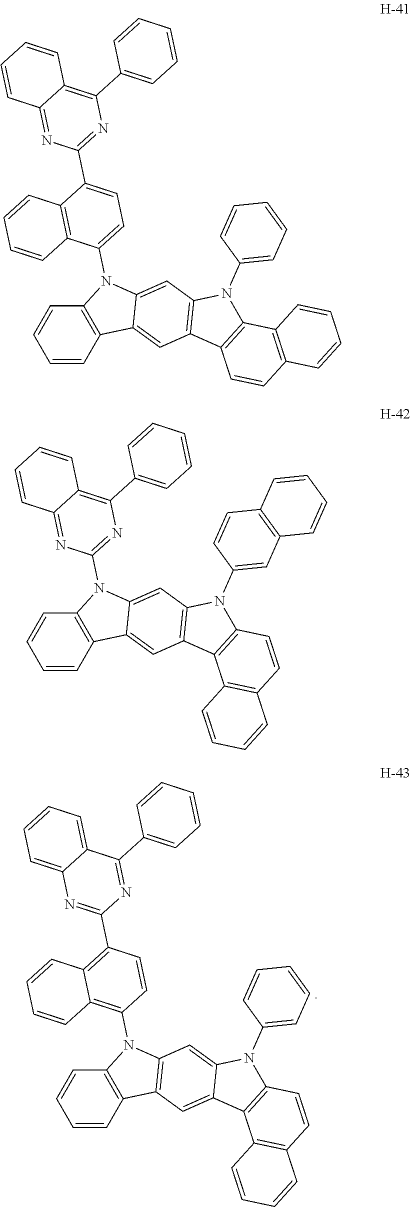 [SMILES: c1ccc(-c2nc(-c3ccc(-n4c5ccccc5c5cc6c7ccc8ccccc8c7n(-c7ccccc7)c6cc54)c4ccccc34)nc3ccccc23)cc1 ; c1ccc(-c2nc(-n3c4ccccc4c4cc5c6c7ccccc7ccc6n(-c6ccc7ccccc7c6)c5cc43)nc3ccccc23)cc1 ; c1ccc(-c2nc(-c3ccc(-n4c5ccccc5c5cc6c7c8ccccc8ccc7n(-c7ccccc7)c6cc54)c4ccccc34)nc3ccccc23)cc1]

7. An organic electroluminescent device comprising the organic electroluminescent material according to claim 1.

8. The organic electroluminescent device according to claim 7, wherein the organic electroluminescent device comprises the compound represented by the formula 1 as a dopant, and the compound represented by the formula 2 as a host.
